(19) United States
(12) Patent Application Publication
(10) Pub. No.: US 2007/0213696 A1
(43) Pub. Date: Sep. 13, 2007

(54) Photocosmetic device

(75) Inventors: Gregory B. Altshuler, Lincoln, MA (US); Ilya Yaroslavsky, North Andover, MA (US); James S. Cho, Westford, MA (US); Stewart Wilson, Billerica, MA (US); Liam O'Shea, Medford, MA (US); Andrey Belikov, St. Petersburg (RU)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: PALOMAR MEDICAL TECHNOLOGIES, INC., Burlington, MA (US)

(21) Appl. No.: 11/415,362

(22) Filed: May 1, 2006

Related U.S. Application Data
(60) Provisional application No. 60/781,083, filed on Mar. 10, 2006.

(51) Int. Cl. A61B 18/18; A61N 5/06

(52) U.S. Cl. 606/9; 607/88; 607/89

(57) ABSTRACT

An apparatus is disclosed for use by a consumer in a non-medical setting that uses at least one low power optical radiation source in a suitable device that can be positioned over a treatment area for a substantial period of time or can be moved over the treatment area one or more times during each treatment. The apparatus can be moved over or applied to or near the consumer's skin surface as light or other electromagnetic radiation is applied to the skin.

### RELATED APPLICATIONS

This application claims benefit of priority to U.S. Provisional Application No. 60/781,083, filed Mar. 10, 2006 entitled Photocosmetic Device. All content disclosed in this application is hereby incorporated by reference in its entirety.

### BACKGROUND OF THE INVENTION

1. Technical Field

This invention relates to methods and apparatus for utilizing electromagnetic radiation, especially radiation with wavelengths between 300 nm and 100 μm, to treat various dermatology, cosmetic, health, and immune conditions, and more particularly to such methods and apparatus operating at power and energy levels that they are safe enough and inexpensive enough to be performed in both medical and non-medical settings, including spas, salons and the home.

2. Background Art

Optical radiation has been used for many years to treat a variety of dermatology and other medical conditions. Currently, photocosmetic procedures are performed using professional-grade devices. Such procedures have generally involved utilizing a laser, flash lamp or other relatively high power optical radiation source to deliver energy to the patient's skin surface in excess of 100 watts/cm², and generally, to deliver energy substantially in excess of this value. The high-power optical radiation source(s) required for these treatments (a) are expensive and can also be bulky and expensive to mount; (b) generate significant heat which, if not dissipated, can damage the radiation source and cause other problems, thus requiring that bulky and expensive cooling techniques be employed, at least for the source; and (c) present safety hazards to both the patient and the operator, for example, to both a person's eyes and non-targeted areas of the patient's skin. As a result, expensive safety features must frequently be added to the apparatus, and generally such apparatus must be operated only by medical personnel. The high energy at the patient's skin surface also presents safety concerns and may limit the class of patients who can be treated; for example, it may often not be possible to treat very dark-skinned individuals. The high energy may further increase the cost of the treatment apparatus by requiring cooling of tissue above and/or otherwise abutting a treatment area to protect such non-target tissue.

The high cost of the apparatus heretofore used for performing optical dermatology procedures, generally in the tens of thousands of dollars, and the requirement that such procedures be performed by medical personnel, has meant that such treatments are typically infrequent and available to only a limited number of relatively affluent patients.

However, a variety of conditions, some of them quite common, can be treated using photocosmetic procedures (also referred to as photocosmetic treatments). For example, such treatments include, but are not limited to, hair growth management, including limiting or eliminating hair growth in undesired areas and stimulating hair growth in desired areas, treatments for PFB (Pseudo Follicolitus Barbe), vascular lesions, skin rejuvenation, skin anti-aging including improving skin texture, pore size, elasticity, wrinkles and skin lifting, improved vascular and lymphatic systems, improved skin moistening, removal of pigmented lesions, repigmentation, tattoo reduction/removal, psoriasis, reduction of body odor, reduction of oiliness, reduction of sweat, reduction/removal of scars, prophylacetic and prevention of skin diseases, including skin cancer, improvement of subcutaneous regions, including reduction of fat/cellulite or reduction of the appearance of fat/cellulite, pain relief, biostimulation for muscles, joints, etc. and numerous other conditions.

Additionally, acne is one of the conditions that are treatable using photocosmetic procedures. Acne is a widely spread disorder of sebaceous glands. Sebaceous glands are small oil-producing glands. A sebaceous gland is usually a part of a sebaceous follicle (which is one type of follicle), which also includes (but is not limited to) a sebaceous duct and a pilary canal. A follicle may contain an atrophic hair (such a follicle being the most likely follicle in which acne occurs), a vellus hair (such a follicle being a less likely follicle for acne to develop in), or may contain a normal hair (acne not normally occurring in such follicles).

Disorders of follicles are numerous and include acne vulgaris, which is the single most common skin affliction. Development of acne usually starts with formation of non-inflammatory acne (comedo) that occurs when the outlet from the gland to the surface of the skin is plugged, allowing sebum to accumulate in the gland, sebaceous duct, and pilary canal. Although exact pathogenesis of acne is still debated, it is firmly established that comedo formation involves a significant change in the formation and desquamation of the keratinized cell layer inside the infrainfundibulum. Specifically, the comedos form as a result of defects in both desquamating mechanism (abnormal cell cornification) and mitotic activity (increased proliferation) of cells of the epithelial lining of the infrainfundibulum.

The chemical breakdown of triglycerides in the sebum, predominantly by bacterial action, releases free fatty acids, which in turn trigger an inflammatory reaction producing the typical lesions of acne. Among microbial population of pilosebaceous unit, most prominent is Propionibacterium Acnes (P. Acnes). These bacteria are causative in forming inflammatory acne.

A variety of medicines are available for acne. Topical or systemic antibiotics are the mainstream of treatment. Oral isotretinoin is a very effective agent used in severe cases. However, an increasing antibiotic resistance of P. Acnes has been reported by several researchers, and significant side effects of isotretinoin limit its use. As a result, the search continues for efficient acne treatments with at most minimal side effects, and preferably with no side effects.

To this end, several techniques utilizing light have been proposed. For example, R. Anderson discloses laser treatments of sebaceous gland disorders with laser sensitive dyes, the method of this invention involving applying a chromophore-containing composition to a section of the skin surface, letting a sufficient amount of the composition penetrate into spaces in the skin, and exposing the skin section to (light) energy causing the composition to become photochemically or photothermally activated. A similar technique is disclosed in N. Kollias et al., which involves exposing the subject afflicted with acne to ultraviolet light having a wavelength between 320 and 350 nm.

P. Papageorgiou, A. Katsambas, A. Chu, Phototherapy with blue (415 nm) and red (660 nm) light in the treatment of acne vulgaris. Br. J. Dermatology, 2000, v. 142, pp. 973-978 (which is incorporated herein by reference) reports using blue (wavelength 415 nm) and red (660 nm) light for phototherapy of acne. A method of treating acne with at least one light-emitting diode operating at continuous-wave (CW) mode and at a wavelength of 660 nm is also disclosed in E. Mendes, G. Iron, A. Harel, Method of treating acne, U.S. Pat. No. 5,549,660. This treatment represents a variation of photodynamic therapy (PDT) with an endogenous photosensitizing agent. Specifically, P. Acnes are known to produce porphyrins (predominantly, coproporphyrin), which are effective photosensitizers. When irradiated by light with a wavelength strongly absorbed by the photosensitizer, this molecule can give rise to a process known as the generation of singlet oxygen. The singlet oxygen acts as an aggressive oxidant on surrounding molecules. This process eventually leads to destruction of bacteria and clinical improvement of the condition. Other mechanisms of action may also play a role in clinical efficacy of such phototreatment.

B. W. Stewart, Method of reducing sebum production by application of pulsed light, U.S. Pat. No. 6,235,016 B1 teaches a method of reducing sebum production in human skin, utilizing pulsed light of a range of wavelengths that is substantially absorbed by the lipid component of the sebum. The postulated mechanism of action is photothermolysis of differentiated and mature sebocytes.

Regardless of the specific technique or procedure that may be employed, treatment of acne with visible light, especially in the blue range of the spectrum, is generally considered to be an effective method of acne treatment. Acne bacteria produce porphyrins as a part of their normal metabolism process. Irradiation of porphyrins by light causes a photosensitization effect that is used, for example, in the photodynamic therapy of cancer. The strongest absorption band of porphyrins is called the Soret band, which lies in the violet-blue range of the visible spectrum (405-425 nm). While absorbing photons, the porphyrin molecules undergo singlet-triplet transformations and generate the singlet atomic oxygen that oxidizes the bacteria that injures tissues. The same photochemical process is initiated when irradiating the acne bacteria. The process includes the absorption of light within endogenous porphyrins produced by the bacteria. As a result, the porphyrins degrade liberating the singlet oxygen that oxidize the bacteria and eradicate the P. acnes to significantly decrease the inflammatory lesion count. The particular clinical results of this treatment are reported (A. R. Shalita, Y. Harth, and M. Elman, “Acne PhotoClearing (APC.™) Using a Novel, High-Intensity, Enhanced, Narrow-Band, Blue Light Source,” Clinical Application Notes, V.9, N1). In clinical studies, the 60% decrease of the average lesion count was encountered when treating 35 patients twice a week for 10 minutes with 90 mW/cm² and dose 54 J/cm² of light from the metal halide lamp. The total course of treatment lasted 4 weeks during which each patient underwent eight treatments.

To date, photocosmetic procedures for the treatment of acne and other conditions have been performed in a dermatologist's office for several reasons. Among these reasons are: the expense of the devices used to perform the procedures; safety concerns related to the devices; and the need to care for optically induced wounds on the patient's skin. Such wounds may arise from damage to a patient's epidermis caused by the high-power radiation and may result in significant pain and/or risk of infection. It would be desirable if methods and apparatus could be provided, which would be inexpensive enough and safe enough that such treatments could be performed by non-medical personnel, and even self-administered by the person being treated, permitting such treatments to be available to a greatly enlarged segment of the world's population.

### SUMMARY OF THE INVENTION

One aspect of the invention is a handheld device for the treatment of acne using radiant energy that has a housing with an aperture, a radiation source mounted in the housing and oriented to transmit radiation through the aperture, and a heat dissipation element mounted in the housing and in thermal communication with the radiation source. The radiation source may be configured to irradiate the tissue with radiation between approximately 10 mW/cm² and approximately 100 W/cm².

Preferred embodiments of this aspect of the invention may include some of the following additional features. The radiation source may be configured to irradiate the tissue with radiation between approximately 100 mW/cm² and approximately 100 W/cm². The radiation source may be configured to irradiate the tissue with radiation between approximately 1 W/cm² and approximately 100 W/cm². The radiation source may be configured to irradiate the tissue with radiation between approximately 10 W/cm² and approximately 100 W/cm².

The aperture may have an area of at least approximately 4 cm². The aperture may have an area of at least approximately 9 c^{m2}. The aperture may have an area of at least approximately 14.44 cm². The aperture may have an area of at least approximately 16 cm².

The radiation source may be configured to provide at least approximately 2.5 W of optical power. The radiation source may be configured to provide at least approximately 5 W of optical power. The radiation source may be configured to provide at least approximately 10 W of optical power.

The handheld device may be a device for self-use by a consumer. The housing may have a head portion containing the aperture and a handle portion to be held by a user. The aperture may include a sapphire window or a plastic window. The radiation source may be a solid state optical radiation source, such as an LED radiation source. The radiation source may be a laser radiation source. The radiation source may be an array of semiconductor elements. The radiation source may be an optical radiation source.

The device may have a first radiation source and a second radiation source capable of generating radiation within different ranges of wavelengths. The radiation sources may also be capable of operating at multiple wavelengths. The first radiation source may be capable of producing radiation independently from the second radiation source.

The handheld device may have a power source configured to supply power in a continuous wave mode, quasi-continuous wave mode, pulsed wave mode, or in other power modes. The sensors may be electrically connected to a controller and configured to provide an electrical signal when corresponding sections of the aperture are in contact with the tissue. The controller may cause the radiation source to be illuminated when the sensor provides the electrical signals.

The device may have multiple radiation sources with corresponding sensors connected to the controller and configured to provide a electrical signals to control each source. The radiation source may be an array of solid state optical radiation sources.

The aperture may be thermally conductive, allowing heat from the radiation source to be transferred to an area of the tissue being treated via the aperture.

The device may also include an alarm electrically connected to the controller to provide an output signal to the alarm to provide information to the user. The alarm may be an audible sound generator. The alarm may be a light-emitting device. The alarm may be configured to alert the user that a treatment time has expired.

Another aspect of the invention is a handheld device for the treatment of acne using radiant energy that has a housing with an aperture, a radiation source oriented to transmit radiation through the aperture, a controller electrically connected to the radiation source, and a sensor electrically connected to the controller. The controller may be configured to provide an output signal in response to an input signal from the sensor, and the radiation source may be configured to irradiate the tissue with radiation between approximately 1 W/cm² and approximately 100 W/cm².

### BRIEF DESCRIPTION OF THE DRAWINGS

Illustrative, non-limiting embodiments of the present invention will be described by way of example with reference to the accompanying drawings, in which the same reference numeral is for the common elements in the various figures, and in which:

FIG. 1 is a front perspective view of a photocosmetic device according to some aspects of the invention;

FIG. 2 is side perspective view of the photocosmetic device of FIG. 1;

FIG. 3 is an exploded view of the photocosmetic device of FIG. 1;

FIG. 4 is a perspective view of an LED module of the photocosmetic device of FIG. 3;

FIG. 5 is an exploded view of the LED module of FIG. 4;

FIG. 6 is a front schematic view of an LED module of the photocosmetic device of FIG. 3;

FIG. 7 is a front schematic view of an optical reflector of the photocosmetic device of FIG. 3;

FIG. 8 is a cross-sectional side view of a portion of an LED module according to aspects of the invention;

FIG. 9 is a back perspective view of a heatsink assembly of the photocosmetic device of FIG. 3;

FIG. 10 is a back perspective view of a portion of a heatsink assembly of the photocosmetic device of FIG. 3;

FIG. 11 is a front perspective view of some interior components of the photocosmetic device of FIG. 3;

FIG. 12 is schematic view of a control system of the photocosmetic device of FIG. 3;

FIG. 13 is a front perspective view of an attachment for use with the photocosmetic device of FIG. 3;

FIG. 13A is a side cross-sectional view of the attachment of FIG. 13;

FIG. 14 is a side view of another example of a embodiment of a photocosmetic device;

FIG. 15 is a front schematic view of another example of an aperture for a photocosmetic device;

FIG. 16 is a front view of another example of a embodiment of a photocosmetic device;

FIG. 17 is an exploded view of an alternate embodiment of a photocosmetic device;

FIG. 18 is a side perspective view of the photocosmetic device of FIG. 17;

FIG. 19 is an exploded view of a pump assembly of the photocosmetic device of FIG. 17;

FIG. 20 is a cross-sectional side view of the pump assembly and a reservoir of the photocosmetic device of FIG. 17;

FIG. 21 is a perspective view of another example of a embodiment of a photocosmetic device;

FIG. 22 is a cross-sectional side view of a portion of the photocosmetic device of FIG. 21;

FIG. 23 is a cross-sectional side view of a portion of the photocosmetic device of FIG. 21;

FIG. 24 is an exploded view of components of a light source of the photocosmetic device of FIG. 21;

FIG. 25 is an exploded view of components of a light source of the photocosmetic device of FIG. 21;

FIG. 26 is a perspective view of a light source of the photocosmetic device of FIG. 21;

FIG. 27 is a schematic illustration of a head of the photocosmetic device of FIG. 21; and

FIG. 28 is a schematic view of an optical window having an abrasive surface.

### DETAILED DESCRIPTION

### Photocosmetic Procedures in a Non-Medical Environment

While certain photocosmetic procedures, such as CO₂ laser facial resurfacing, where the entire epidermal layer is generally removed, will likely continue for the time being to be performed in the dermatologist's office for medical reasons (e.g., the need for post-operative wound care), there are a large number of photocosmetic procedures that could be performed by a consumer in a non-medical environment (e.g., the home) as part of the consumer's daily hygienic regimen, if the consumer could perform such procedures in a safe and effective manner using a cost-effective device. Photocosmetic devices for use by a consumer in a non-medical environment may have one or more of the following characteristics: (1) the device preferably would be safe for use by the consumer, and should avoid injuries to the body, including the eyes, skin and other tissues; (2) the device preferably would be easy to use to allow the consumer or other operator to use the device effectively and safely with minimal training or other instruction; (3) the device preferably would be robust and rugged enough to withstand abuse; (5) the device preferably would be easy to maintain; (6) the device preferably would be relatively inexpensive to manufacture and would be capable of being mass-produced; (7) the device preferably would be small and easily stored, for example, in a bathroom; and (8) the device preferably would have safety features standard for consumer appliances that are powered by electricity and that are intended for use, e.g., in a bathroom. Currently available photocosmetic devices have limitations related to one or more of the above challenges.

However, there are technical challenges associated with creating such devices for use by a consumer in a non-medical environment, including safety, effectiveness of treatment, cost of the device and size of the device.

### Low-Power Optical Radiation

The invention generally involves the use of a low-power optical radiation source, or preferably an array of low power optical radiation sources, in a suitable head which is either held over a treatment area for a substantial period of time, i.e. one second to one hour, or is moved over the treatment area a number of times during each treatment. Depending on the area of the person's body and the condition being treated, the cumulative dwell time over an area during a treatment will vary. The treatments may be repeated at frequent intervals, i.e. daily, or even several times a day, weekly, monthly or at other appropriate intervals. The interval between treatments may be substantially fixed or may be on an “as required” basis. For example, the treatments may be on a substantially regular or fixed basis to initially treat a condition, and then be on as an “as required” basis for maintenance. Treatment can be continued for several weeks, months, years and/or can be incorporated into a user's regular routine hygiene practices. Certain treatments are discussed further in U.S. application Ser. No. 10/740,907, entitled “Light Treatments For Acne And Other Disorders Of Follicles,” filed Dec. 19, 2003, which is incorporated herein by reference.

Thus, while light has been used in the past to treat various conditions, such treatment has typically involved one to ten treatments repeated at widely spaced intervals, for example, weekly, monthly or longer. By contrast, the number of treatments for use with embodiments according to aspects of this invention can be from ten to several thousand, with intervals between treatments from several hours to one week or more. It is thought that, for certain conditions such as acne or wrinkles, multiple treatments with low power could provide the same effect as one treatment with high power. The mechanism of treatment can include photochemical, photo-thermal, photoreceptor, photo control of cellular interaction or some combination of these effects. For multiple systematic treatments, a small dose of light can be effective to adjust cell, organ or body functions in the same way as systematically using medicine.

Instead of using single or few treatments of intense light, which must be performed in a supervised condition such as a medical office, the same reduction of the bacteria population level can be reached using a greater number of treatments of significantly lower power and dose using, for example, a hand-held photocosmetic device in the home. Using a relatively lower power treatment, a consumer can use the photocosmetic device in the home or other non-medical environment.

The specific light parameters and formulas of assisted compounds suggested in the present invention provide this treatment strategy. These treatments may preferably be done at home, because of the high number of treatments and the frequent basis on which they must be administered, for example daily to weekly. (Of course, some embodiments of the present invention could additionally be used for therapeutic, instructional or other purposes in medical environments, such as by physicians, nurses, physician's assistants, physical therapists, occupational therapists, etc.)

Depending on the treatment to be performed, the light source may be configured to emit at a single wavelength, multiple wavelengths, or in one or more wavelength bands. The light source may be a coherent light source, for example a ruby, alexandrite or other solid state laser, gas laser, diode laser bar, or other suitable laser light source. Alternatively, the source may be an incoherent light source for example, an LED, arc lamp, flash lamp, fluorescent lamp, halogen lamp, halide lamp or other suitable lamp.

Various light based devices can be used to deliver the required light doses to a body. The optical radiation source(s) utilized may provide a power density at the user's skin surface of from approximately 1 mwatt/cm² to approximately 100 watts/cm², with a range of 10 mwatts/cm² to 10 watts/cm² being preferred. The power density employed will be such that a significant therapeutic effect can be achieved, as indicated above, by relatively frequent treatments over an extended time period. The power density will also vary as a function of a number of factors including, but not limited to, the condition being treated, the wavelength or wavelengths employed and the body location where treatment is desired, i.e., the depth of treatment, the user's skin type, etc. A suitable source may, for example, provide a power of approximately 1-100 watts, preferably 2-10 W.

Suitable sources include solid state light sources such as:

1. Light Emitting Diodes (LEDs)—these include edge emitting LED (EELED), surface emitting LED (SELED) or high brightness LED (HBLED). The LED can be based on different materials, such as, without limitation, GaN, AlGaN, InGaN, AlInGaN, AlInGaN/AlN, AlInGaN (emitting from 285 nm to 550 nm), GaP, GaP:N, GaAsP, GaAsP:N, AlGaInP (emitting from 550 nm to 660 nm) SiC, GaAs, AlGaAs, BaN, InBaN, (emitting in near infrared and infrared). Another suitable type of LED is an organic LED using polymer as the active material and having a broad spectrum of emission with very low cost.

2. Superluminescent diodes (SLDs)—An SLD can be used as a broad emission spectrum source.

3. Laser diodes (LD)—A laser diode may be the most effective light source (LS). A wave-guide laser diode (WGLD) is very effective but is not optimal due to the difficulty of coupling light into a fiber. A vertical cavity surface emitting laser (VCSEL) may be most effective for fiber coupling for a large area matrix of emitters built on a wafer or other substrate. This can be both energy and cost effective. The same materials used for LED's can be used for diode lasers.

4. Fiber laser (FL) with laser diode pumping.

5. Fluorescence solid-state light source with electric pumping or light pumping from LD, LED or current/voltage sources (FLS). An FLS can be an organic fiber with electrical pumping.

Other suitable low power lasers, mini-lamps or other low power lamps or the like may also be used as light source(s) in embodiments of the present invention.

LED's are the currently preferred radiation source because of their low cost, the fact that they are easily packaged, and their availability at a wide range of wavelengths suitable for treating various tissue conditions. In particular, Modified Chemical Vapor Deposition (MCVD) technology may be used to grow a wafer containing a desired array, preferably a two-dimensional array, of LED's and/or VCSEL at relatively low cost. Solid-state light sources are preferable for monochromatic applications. However, a lamp, for example an incandescent lamp, fluorescent lamp, micro halide lamp or other suitable lamp is a preferable light source for applying white, red, near infrared, and infrared irradiation during treatment.

Since the efficiency of solid-state light sources is 1-50%, and the sources are mounted in very high-density packaging, heat removal from the emitting area is generally the main limitation on source power. For better cooling, a matrix of LEDs or other light sources can be mounted on a diamond, sapphire, BeO, Cu, Ag, Al, heat pipe, or other suitable heat conductor. The light sources used for a particular apparatus can be built or formed as a package containing a number of elementary components. For improved delivery of light to skin from a semiconductor emitting structure, the space between the structure and the skin can be filled by a transparent material with a refractive index in the range 1.3 to 1.8, preferably between 1.35 and 1.65, without air gaps.

An example of a condition that is treatable using an embodiment of the present invention is acne. In one aspect, the treatment described involves the destruction of the bacteria (P. acnes) responsible for the characteristic inflammation associated with acne. Destruction of the bacteria may be achieved by targeting porphyrins stored in P. Acnes. Porphyrines, such as protoporphyrins, coproporphyrins, and Zn-protoporphyrins are synthesized by anaerobic bacteria as their metabolic product. Porphyrines absorb light in the visible spectral region from 400-700 nm, with strongest peak of absorption in the range of 400-430 nm. By providing light in the selected wavelength ranges in sufficient intensity, photodynamic process is induced that leads to irreparable damage to structural components of bacterial cells and, eventually, to their death. In addition, heat resulting from absorption of optical energy can accelerate death of the bacteria. For example, the desired effect may be achieved using a light source emitting light at a wavelength of approximately 405 nm using an optical system designed to irradiate tissue 0.2-1 mm beneath the skin surface at a power density of approximately 0.01-10 W/cm² at the skin surface. In another aspect of the invention, the treatment can cause resolution or improvement in appearance of acne lesion indirectly, through absorption of light by blood and other endogenous tissue chromophores.

### A Photocosmetic Device for the Treatment of Acne and Other Skin Conditions

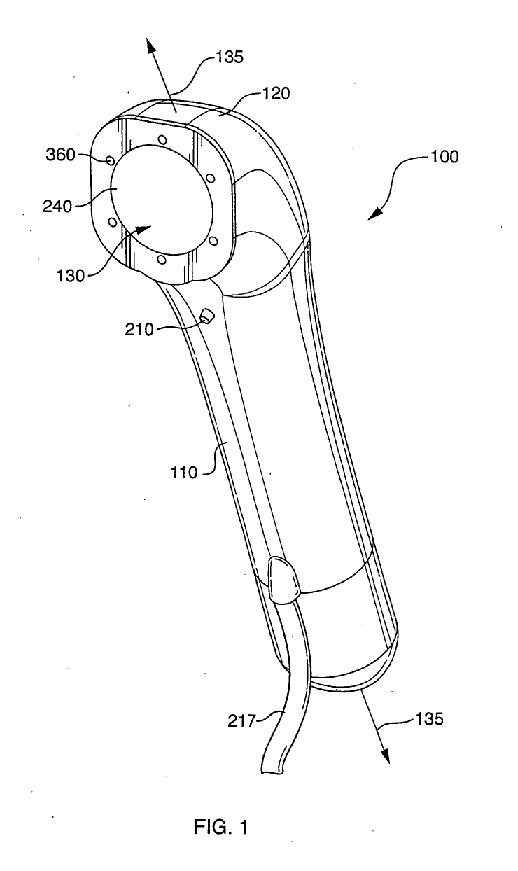

A photocosmetic device according to some aspects of the invention that is designed to treat, for example, acne is described with reference to FIGS. 1 through 3. Photocosmetic device 100 is a device that may be used by a consumer or user, e.g., in the home as part of the consumer's or user's daily hygienic regimen. In this embodiment, photocosmetic device 100 is a hand-held unit that: is approximately 52 mm in width; 270 mm in length; has a total internal volume of approximately 307 cc; and has a total weight of approximately 370 g.

Preferably, photocosmetic device 100 comes with simple and easy-to-follow instructions that instruct the user how to use photocosmetic device 100 both safely and effectively. Such instructions may be written and may include pictures and/or such instructions may be provided through a visible medium such as a videotape, DVD, and/or Internet.

Generally, photocosmetic device 100 includes proximal and distal portions 110 and 120 respectively. Proximal portion 110 serves as a handle that allows the user to grasp the device and administer treatment. Distal portion 120 is referred to as the head of photocosmetic device 100 and includes an aperture 130 that allows light produced by photocosmetic device 100 to illuminate the tissue to be treated when aperture 130 is placed in contact with or near the surface of the tissue to be treated. Generally, to treat acne, the user would place the aperture 130 of photocosmetic device 100 on their skin to administer treatment.

When viewed from the front of photocosmetic device 100, distal portion 120 flares outward to be slightly wider than proximal portion 110. When viewed from the side of photocosmetic device 100, distal portion 120 curves to orient aperture 130 to approximately a 45 degree angle relative to a longitudinal axis 135 extending through proximal portion 110. Of course, this angle may be different in other embodiments to potentially improve the ergonomics of the device. Alternatively, an embodiment may include an adjustable or movable head that pivots in various directions, such as up and down to increase or decrease the relative angle of the aperture relative to the longitudinal axis of proximal portion 110 and/or that swivels or rotates about the longitudinal axis of proximal portion 110.

Photocosmetic device 100 is designed to meet the specifications listed below in Table 1. As noted above, the embodiment described as photocosmetic device 100 has a weight of approximately 370 g, which has been determined to accommodate enough coolant to provide for a total treatment time of approximately 10 minutes. An alternative embodiment similar to photocosmetic device 100 would weigh approximately 270 g and accommodate a total treatment time of approximately 5 minutes. Similarly, other embodiments can include more or less coolant to increase or decrease available treatment time.

TABLE 1
Device Specifications for an Embodiment of a Photocosmetic Device for Treating Acne.

| TARGET Specification | Symbol | Value | Units |
|---|---|---|---|
| Total Optical Power | Ptot | 5 | W |
| Dominant Wavelength | | 400-430 | nm |
| Spot Size Diameter | SS | 38 (1.5) | mm (in) |
| Operation Time | Top | 5 | Min |
| Lifetime | Tlife | 100 | Hrs |
| Mode of Operation (Power) | MODE | QCW or CW | |
| Pulse Width | PW | 100 ms < PW < CW | mSec |
| Duty Cycle | DC | 10 < DC < 100 | % |
| Target Handpiece Weight | Wmax | 270 | grams |
| Maximum Exposure Level | MEL | 140 | W/m²/sr/nm |
| Maximum Exposure Time | MET | 60 | min |
| Maximum Operating Voltage | Vmax | 26 | V |
| Maximum Operating Current | Imax | 4 | A |
| Maximum Heat Load | Hmax | 87 | W |
| MAX Allowable Coolant Temperature | Tcmax | 70 | ° C. |
| Max External Window Temperature | Tskin | 35 | ° C. |
| Max Allowable Handpiece External | Thp max | 50 | ° C. |
| Temp | | | |
| Max Ambient Temperature | Tamax | 30 | ° C. |
| Minimum Coolant Volume | Cvol | 180 | cc |
| Maximum Optical Loss | Oloss | 10 | % |

In Table 1, where “maximum,” “minimum,” “total” and similar terms are used, they are meant for a particular embodiment.

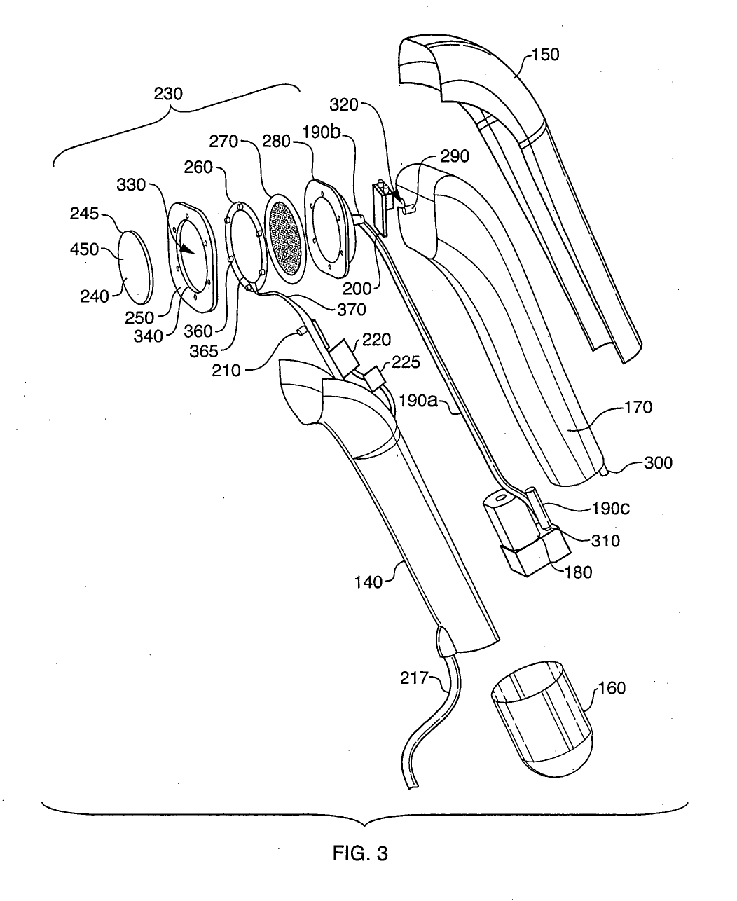

As shown in FIG. 3, photocosmetic device 100 includes a front housing section 140, a back housing section 150, and a bottom housing section 160. Housing sections 140, 150 and 160 fit together along the edges of each section to form a housing for photocosmetic device 100. Within the housing, photocosmetic device 100 includes a coolant reservoir 170, a pump 180, coolant tubes 190a-190c, a thermal switch 200, a power control switch 210, electronic control system 220, a boost chip 225, and a light source assembly 230.

### Light Source Assembly

Light source assembly 230 includes a number of components: window 240, window housing 250, contact sensor ring 260, LED module 270, and heatsink assembly 280. As will be appreciated from FIG. 3, when the three housing sections 140, 150 and 160 are assembled, they form an opening in the distal portion 120 of photocosmetic device 100. That opening accommodates light source assembly 230, which is secured within the opening to form a face of distal portion 120 used to treat tissue, when light source assembly 230 is assembled.

The components of light source assembly 230 are secured in close proximity to one another in the order shown in FIG. 3 to form light source assembly 230, and are secured using screws to hold them in place. Window 240 is secured within an opening of window housing 250, which forms aperture 130. Contact sensor ring 260 is secured directly behind and adjacent to window housing 250 within the interior housing of photocosmetic device 100. Six contact sensors 360 are located equidistantly around the window 240. Window housing 250 includes six small openings 350 directly adjacent to, and evenly spaced about, opening 330 to accommodate contact sensors 360 of contact sensor ring 260. Contact sensor ring 260 is placed directly adjacent to window housing 250 such that the contact sensors 360 extend through the openings 350—each of six contact sensors 360 fitting into one of each of the six corresponding openings 350. LED module 270 is secured directly behind and adjacent to contact sensor ring 260. Similarly, heatsink assembly 280 is secured directly behind and adjacent to LED module 270.

Window 240 is secured within a circular opening 330 of window housing 250 along the edge 340 of the opening 330. Light is delivered through window 240, which forms a circularly symmetric aperture having a diameter of 38 mm (1.5″). Although window 240 is shown as a circle, various alternate shapes can be used. Window 240 is made of sapphire, and is configured to be placed in contact with the user's skin. Sapphire is used due to its good optical transmissivity and thermal conductivity. The sapphire window 240 is substantially transparent at the operative wavelength, and is thermally conductive to remove heat from a treated skin surface.

In alternative embodiments, sapphire window 240 may be cooled to remove heat from the sapphire element and, thus, remove heat from skin placed in contact with sapphire window 240 during treatment. In addition, other embodiments could employ materials other than sapphire also having good optical transmissivity and heat transfer properties, such as mineral glass, dielectric crystal such as quartz or plastic. For example, to save cost and reduce weight, window 240 could be an injection molded optical plastic material.

Optionally, prior to treatment with the photocosmetic device, a lotion that is transparent at the operative wavelength(s) may be applied on the skin. Such a lotion may improve both optical transmissivity and heat transfer properties. In still other embodiments, the lateral sides 245 of the window housing can be coated with a material reflective at the operative wavelength (e.g., copper, silver or gold). Additionally, the outer surface of window housing 250 or any other surface exposed to light which is reflected or scattered back from the skin may be reflective (e.g., coated with a reflective material) to re-reflect such light back to the area of tissue being treated. This is referred to as “photon recycling” and allows for more efficient use of the power supplied to light source assembly 230, thereby reducing the relative amount of heat generated by source assembly 230 per the amount of light delivered to the tissue. Any such surface could be made to be highly reflective (e.g., polished) or could be either coated or covered with a suitable reflective material (e.g., vacuum deposition of a reflective material or covered with a flexible silver-coated film).

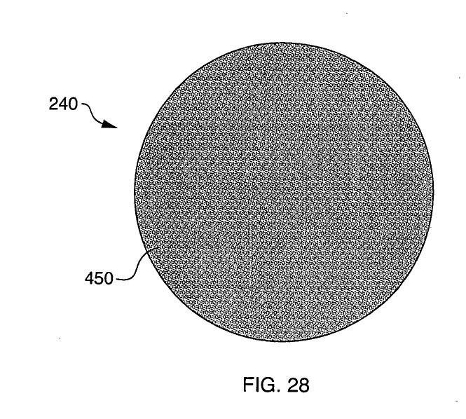

Referring also to FIG. 28, window 240 preferably has a micro-abrasive surface 450 located on the exterior of photocosmetic device 100. Micro-abrasive surface 450 has a micro surface roughness between 10 and 500 microns peak to peak, preferably 60+/−10 microns peak to peak. However, many other configurations are possible, including variations on the dimensions of the surface and the pattern and shape of the abrasive portions of the surface, e.g., employing rib-shaped structures, teeth-like structures, and structures that are arranged in circular pattern. Preferably, the micro-abrasive surface 450 includes small sapphire particles adhered to window 240 or to reduce costs, the particles can be made of plastic. Moving the micro-abrasive surface 450 against the skin provides removal of dead skin cells from the stratum corneum which can stimulate the normal healing/replacement process of the stratum corneum as described in more detail below.

Additionally, the micro-abrasive surface need not be a window. Alternatively, for example, an abrasive surface, including a micro-abrasive surface, may be placed about the circumference of an aperture of a photocosmetic device or may be placed adjacent to the aperture or window. Moreover, the micro-abrasive surface, whether configured as a window, adjacent to a window, or otherwise configured, may be replaceable. Thus, a worn abrasive surface may be replaced with a new abrasive surface to maintain performance of the device over time.

Contact sensor ring 260 provides contact sensors 360 for detecting contact with tissue (e.g., skin). Contact sensor ring 260 can be used to detect when all of or portions of window 240 are in contact with, or in close proximity to, the tissue to be treated. In one embodiment, contact sensors 360 are e-field sensors. In alternative embodiments, other sensor technologies, such as optical (LED or laser), impedance, conductivity, or mechanical sensors can be used. The contact sensors can be used to ensure that no light is emitted from photocosmetic device 100 (e.g., no LEDs are illuminated) unless all of the sensors detect simultaneous contact with tissue. Alternatively, and preferably for highly contoured surfaces, such as the face, contact sensors 360 can be used to ensure that only LEDs in certain portions of LED module 270 are illuminated. For example, if only a portion of window 240 is in close proximity to or in contact with skin or other tissue, only certain contact sensors will detect contact with skin and such limited contact can be used to illuminate only those LEDs corresponding to those sensors. This is referred to as “intelligent contact control.”

In the embodiment shown, contact sensors 360 are mounted equidistantly about a ring 365, which is composed of electronic circuit board or other suitable material. LED module 270, which is described in greater detail below, is mounted directly behind and adjacent to contact sensor ring 260. The six contact sensors 360 are electrically connected to electronic control system 220 via electrical connector 370. In alternative embodiments, more or fewer contact sensors may be used and they may not be mounted equidistantly or in a ring.

As described above, contact sensor ring 260 is secured to the interior surface of window housing 250 such that the sensors extend through holes in housing 250 to allow the contact sensors to be able to directly contact tissue. In this embodiment, the contact sensors are used to detect when the window 240, including micro-abrasive surface 450, is in contact with the skin.

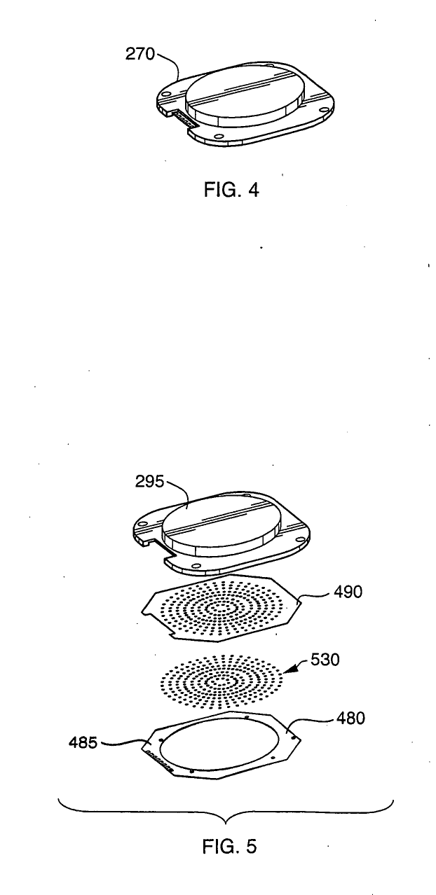

Referring to FIGS. 4-6, LED module 270 includes an array of LED dies 530 (shown in FIG. 5), which generate light when powered by photocosmetic device 100. LED module 270 delivers approximately 4.0 W of optical power, which is emitted in, for example, the 400 to 430 nm (blue) wavelength region. This range is known in the art to be safe for the treatment of skin and other tissue. Optical power is evenly distributed across the aperture with less than 10% power variation.

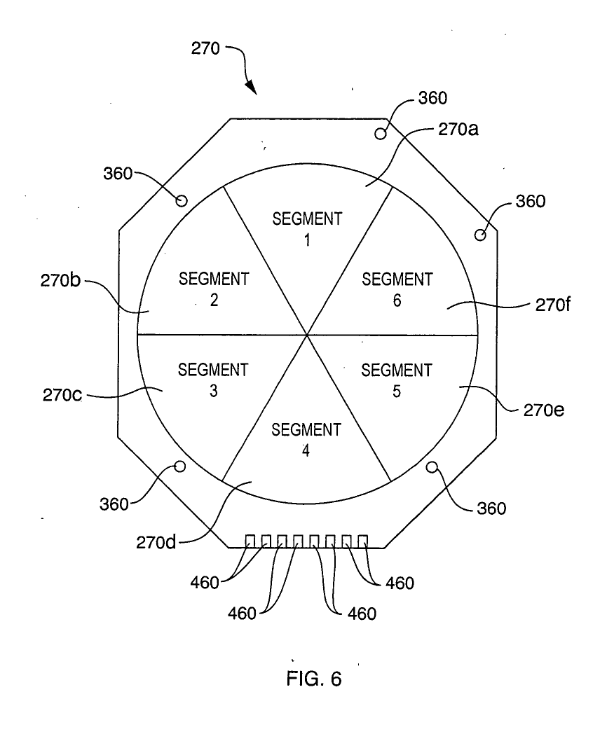

In one embodiment, LED module 270 is divided conceptually and electrically into six pie-shaped sections 270a-270f roughly equal in size and amount of illumination provided. This allows photocosmetic device 100, using electronic control system 220, to illuminate only certain of the pie-shaped segments 470a-470f in certain treatment conditions. Each of the six contact sensors 360 is aligned with and corresponds to one of the pie-shaped segments 470a-470f (as shown in FIG. 6). Thus, the control electronics may illuminate certain segments depending upon contact detected by one or more contact sensors. In alternate embodiments, various shapes can be used for the segments and the segments can be different in size, shape and optical power. In addition, multiple contact sensors may be associated with each segment and each sensor may be associated with one or more segments.

Referring to FIG. 5, the substrate 480 of LED module 270/LED segments 470a-470f can be made of any highly thermally conductive and electrically resistive ceramic. The individual LED dies 530 are mounted to substrate 480. The surface 485 of substrate 480, to which the LED dies 530 are attached, is pattern metallized to accommodate the total number of LEDs as specified in Table 2 below. Each individual LED die 530 should be attached with a suitable robust die attach material to minimize thermal resistance. The pattern metal should be capable of being heated to 325 degrees C. for a period of 15 minutes. In addition, the backside (opposite of the side shown FIG. 5) also is pattern metallized as well to provide appropriate electrical connections. The substrate of LED module 270 contains a ceramic material that preferably has a thermal conductivity >180 W/m-K and is electrically resistant. The coefficient of thermal expansion for the substrate should be between 3 and 8 ppm/C.

In the embodiment shown, each of the LED segments 470a-470f contains approximately the same number of LEDs, and the power requirement for each section is shown in the following table.

TABLE 2
LED Module Electro-Optical Requirements

| | | # | | Vtot | Itot | | Po |
|---|---|---|---|---|---|---|---|
| SEGMENT | # Series | Parallel | # LED | (V) | (A) | Pe (W) | (W) |
| 1 | 5 | 8 | 40 | 24.84 | 0.568 | 14.11 | 0.84 |
| 2 | 5 | 9 | 45 | 24.84 | 0.639 | 15.87 | 0.95 |
| 3 | 5 | 9 | 45 | 24.84 | 0.639 | 15.87 | 0.95 |
| 4 | 5 | 8 | 40 | 24.84 | 0.568 | 14.11 | 0.84 |
| 5 | 5 | 9 | 45 | 24.84 | 0.639 | 15.87 | 0.95 |
| 6 | 5 | 9 | 45 | 24.84 | 0.639 | 15.87 | 0.95 |
| | | TOTAL | 260 | 24.84 | 3.69 | 91.709 | 5.46 |

LED Module 270 can be powered in continuous-wave (CW), quasi-continuous-wave (QCW), or pulsed (P) mode. The term “quasi-CW” refers to a mode when continuous electrical power to the light source(s) is periodically interrupted for controlled lengths of time. The term “pulsed” refers to a mode when the energy (electrical or optical) is accumulated for a period of time with subsequent release during a controlled length of time. Optimal choice of the temporal mode depends on the application. Thus, for photochemical treatments, the CW or QCW mode can be preferable. For photothermal treatment, pulsed mode can be preferable. The temporal mode can be either factory-preset or selected by the user. For treatment of acne, CW or QCW modes are preferred, with the duty cycle between 10 and 100% and “on” time between 10 ms and CW. The CW and QCW light sources are typically less expensive than pulsed sources of comparable wavelength and energy. Thus, for cost reasons, it may be preferable to use a CW or QCW source rather than a pulsed source for treatments.

For the treatment of acne, and for many other treatments, quasi-continuous operation to power the LED die 530 of LED module 270 is preferred. In the QCW mode of operation, maximum average power can be achieved from the LED. However, the light sources employed may also be operated in continuous wave (CW) mode or pulsed mode. Preferably, appropriate safety measures are incorporated into the design of the photocosmetic device regardless of the mode(s) that is (are) used.

Power is supplied to the LED module 270 via electrical connector 370, which is an electrical flex cable that is attached from the electronic control system 220 to pin connectors 460. The illumination of the LED dies 530 associated with the respective segments 470a-470f is controlled by electronic control system 220. Each segment 470a-470f is controlled separately through one of the independent pin connectors 460, which are located at the bottom of substrate 480. There are eight pin connectors 460, each providing an electrical connection between electronic control system 220 and LED module 270. Read from left to right in FIG. 6, each electrical pin connector provides an electrical connection as follows: (1) ground/cathode; (2) LED segment 470a; (3) LED segment 470b; (4) LED segment 470c; (5) LED segment 470d; (6) LED segment 470e; (7) LED segment 470f; and (8) ground/cathode. Each segment 470a-470f shares a common cathode, but has a separate anode trace from the pin connector 460 to the corresponding segment 470a-470f and back to the common cathode to complete the circuit. Thus, via pin connectors 460, each of the six LED segments 470a-470f can be controlled independently.

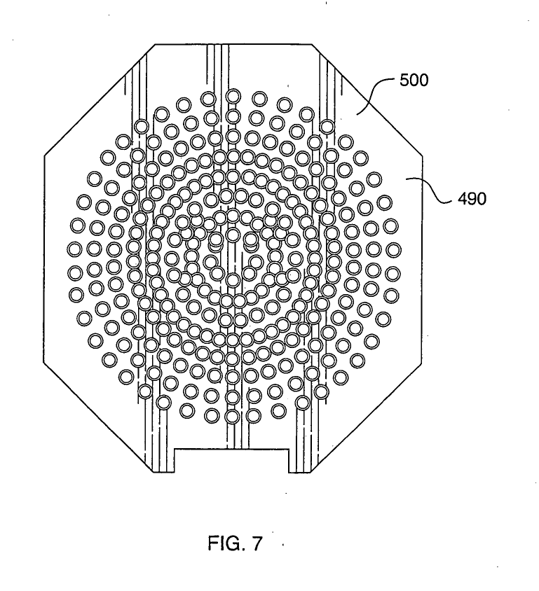

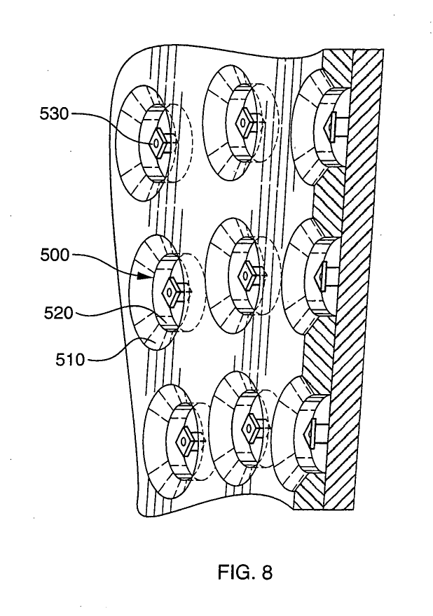

Referring to FIGS. 7 and 8, LED module 270 includes a reflector 490 that is capable of reflecting 95% or more of the light emitted from the LED die 530 of LED module 270. Reflector 490 contains an array of holes 500. Each hole 500 is funnel-shaped having a cone-shaped section 510 and a tube-shaped section 520. Each of the holes 500 of optical reflector 490 correspond to one of the LED dies that are mounted on substrate 480. Thus, when assembled, as shown in FIG. 8, each hole 500 accommodates one LED. Ninety-five percent or more of the light emitted by an LED die that impacts the cone-shaped section 510 within which it is mounted will be reflected toward the tissue to be treated. In addition, reflector 490 provides photon recycling, in that light that is reflected or scattered back from the skin and impacts reflector 490 will be re-reflected back toward the tissue to be treated.

In one embodiment, reflector 490 is made of silver-plated OHFC copper, but can be of any suitable material provided it is highly reflective on all surfaces on which light may impact. More specifically, the surfaces within the holes 500 and the top most surface of reflector 490 facing the window 240 are silver-plated to reflect and/or return light onto the tissue to be treated.

The assembly process for LED module 270 is illustrated with reference to FIG. 5. First, optical reflector 490 is attached to a patterned metallized ceramic substrate 480. Second, the individual LED dies 530 are mounted to substrate 480 through the holes 500 in optical reflector 490. The material used to attach each LED die 530 to substrate 480 should be suitable for minimizing chip thermal resistance. A suitable solder could be eutectic gold tin and this could be pre-deposited on the LED die at the manufacturer. Third, the LED dies 530 are Au wire bonded to provide electrical connections. Finally, the LED dies 530 are encapsulated with the appropriate index matching silicon gel and an optic is added to complete encapsulation 295.

Because the light is delivered through window 240, the LED dies 530 of LED module 270 should be encapsulated and their indexes should be closely matched with the optical component window 240, whether sapphire, an optical grade plastic or other suitable material. In this particular embodiment, the individual LEDs of LED module 270 are manufactured by CREE—the MegaBright LED C405 MB290-S0100. These LEDs have physical characteristics that are suitable for use with window 240 and produce light at the desired 405 nm wavelength.

### Cooling System

Referring to FIG. 3, to prevent light source assembly 230 and other components of photocosmetic device 100 from overheating, photocosmetic device 100 has a cooling system that includes coolant reservoir 170, pump 180, coolant tubes 190a-190c, thermal switch 200, and a heatsink assembly 280.

When light source assembly 230 and heatsink assembly 280 are fully assembled and installed in photocosmetic device 100, thermal switch 200 is mounted directly adjacent to, and in contact with heatsink assembly 280. In the present embodiment, thermal switch 200 is a disc momentary switch manufactured by ITT Industries (part number EDSSC1). To prevent overheating of photocosmetic device 100 during operation, thermal switch 200 monitors the temperature of light source assembly 230. If thermal switch 200 detects excessive temperature, it cuts the power to light source assembly 230 and photocosmetic device 100 will cease to function until the temperature reaches an acceptable level. In one embodiment, the switch shuts off power to photocosmetic device 100, if it detects a temperature of 70° C. or more. Alternatively, a thermal switch could cut power to the light source only and the device could continue to supply power to operate a cooling system to reduce the excessive temperature as quickly as possible.

The cooling system of photocosmetic device 100 further includes a circulatory system to cool the device by removing heat generated in light source assembly 230 during operation. The cooling system could additionally be used to remove heat from window 240. The circulatory system of photocosmetic device 100 includes pump 180, coolant tubes 190a-190c, coolant reservoir 170 and heatsink assembly 280. The coolant reservoir 170 contains an internal space that holds approximately 180 cc of water. When photocosmetic device 100 is in use, the water is circulated by pump 180. Pump 180 is a Micro-Diaphragm Liquid Pump, Single Head OEM Installation Model with DC Motor, model number NF5RPDC-S. The weight, size, and performance of the pump are selected to be suitable for the application, and will vary depending on, for example, the output power of the light source, the volume of coolant, and the total treatment time desired.

Tube 190a is connected at one end to pump 180 and at a second end to heatsink assembly 280. As shown in FIG. 3, tube 190a runs along a groove 320 that extends along the exterior of coolant reservoir 170 to accommodate tube 190a. Tube 190b is connected at one end to heatsink assembly 280 and at a second end to connector port 290 of coolant reservoir 170. Tube 190c is connected at one end to a connector port 300 of coolant reservoir 170 and at a second end to a connector port 310 of pump 180. Each of the coolant tubes 190a-190c are flexible PVC tubing having an inner diameter of 0.125″ and an outer diameter of 0.25″. The tubing has a maximum temperature capacity of 90° C. Each of the six ends of coolant tubes 190a-190c are connected to similar connector ports. However, in FIG. 3, only connector ports 290, 300 and 310 are shown. After the ends of tubes 190a-190c are connected to the respective connector ports, the tubes are sealed to the connector ports to prevent leakage using a commercial grade sealant that is appropriate for this purpose.

When tubes 190a-190c are fully connected, they form a continuous circuit through which a fluid, in this case water, can circulate to cool light source assembly 230. When photocosmetic device 100 is in operation, water preferably flows from coolant reservoir 170, through tube 190c, into pump 180, which forces the fluid through tube 190a, through heatsink assembly 280, through tube 190b and back into coolant reservoir 170.

During operation of photocosmetic device 100, the water flows across heatsink assembly 280 to remove the heat generated by light source assembly 230. Coolant reservoir 170 acts as an additional heatsink for the heat removed from light source assembly 230. By directing the water directly from heatsink assembly 280, through coolant tube 190b and into coolant reservoir 170, the recently heated water is dispersed into coolant reservoir 170, which allows the heat to be dispersed more efficiently than if the recently heated water were first circulated through pump 180. However, the water could flow in either direction in other embodiments.

In generating 5 Watts of optical power, LED module 270 will produce approximately 84-86W of power. The cooling system of photocosmetic device 100 maintains the operating junction temperature below 125 degrees C. for the required treatment time, 10 minutes for this embodiment. The total thermal resistance (Rth) of the junction between the surface of heatsink assembly 280 and the water contained within the circulatory system is approximately 0.315 K/W. Therefore, the junction temperature rise relative to the water temperature is approximately 26.5° C. (0.315 C/W×84 W). The maximum operating junction temperature (T_{juction}) for the individual LED dies 530 is 125° C. The junction temperature is given by the following formula:
Tj=(Rₜₕ×HL)+Ta+ΔTᵣᵢₛₑ

Where ΔTᵣᵢₛₑ is the temperature increase of the water as heat is expelled into it. Therefore, if Tj max is 125° C. and the ambient temperature is 30° C., the maximum water temperature rise should be no greater than:
ΔTᵣᵢₛₑ=125° C.−26° C.−30° C.=69° C.

Therefore, in this embodiment, Ta preferably is limited to <70° C. during operation. This value will change depending on the embodiment, and may not be applicable to other embodiments using different types of cooling systems, as discussed below.

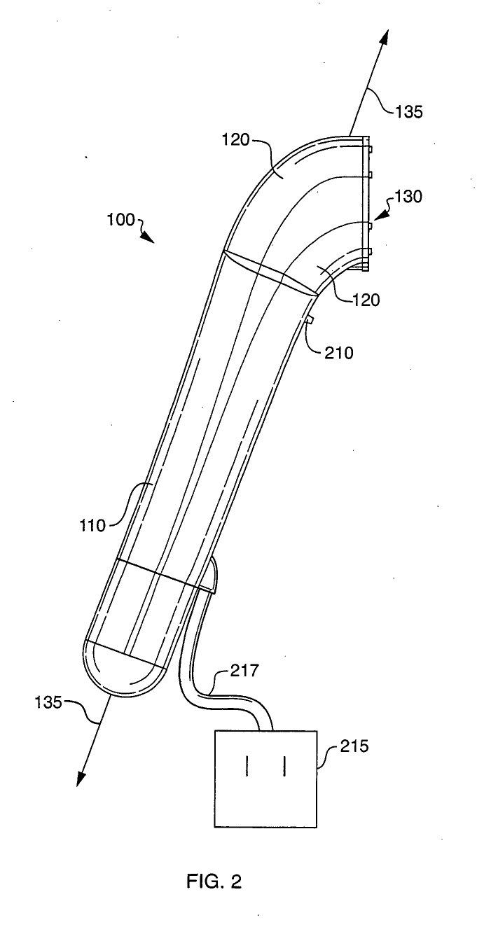

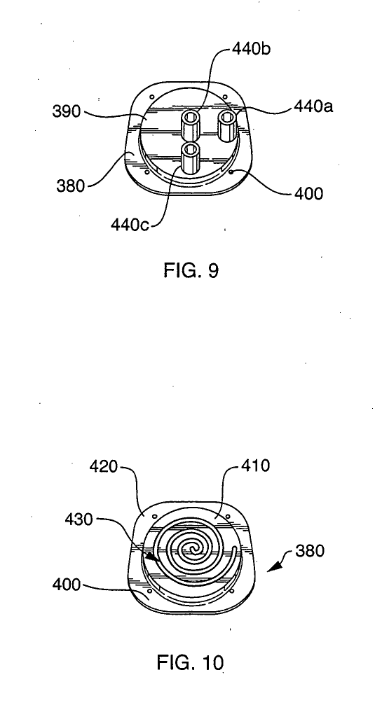

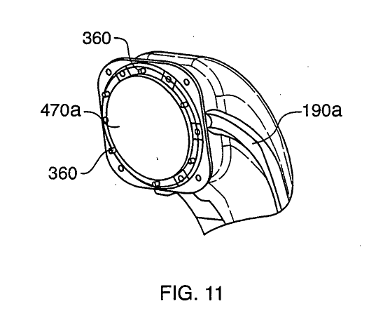

Referring to FIGS. 9 and 10, the heatsink assembly 280 is shown in greater detail. Heatsink assembly 280 preferably is made of copper, but can alternatively be made of other thermally conductive metals or other materials suitable to serve as heatsinks. Heatsink assembly 280 consists of a face plate 380 and a backplate 390. Face plate 380 contains four holes 400 that are used to secure the heatsink assembly 280 within light source assembly 230. When heatsink assembly 280 is secured in place, a forward or distally facing surface of faceplate 380 is in contact with the backward or proximally facing surface of LED module 270 (as shown in FIG. 2). (Note that the distally facing surface of face plate 380 is facing downward in both FIGS. 9 and 10, and, thus, cannot be seen in those figures.) During operation of photocosmetic device 100, the contact between the distally facing surface of faceplate 380 and the back of LED module 270 facilitates the transfer of heat from LED module 270 to heatsink assembly 280.

The backward or proximally facing surface of faceplate 380, shown in FIG. 10, includes a raised portion 410. Raised portion 410 is relatively thicker than the outer edge 420 of faceplate 380 and is circular—being located in the geographic center of surface 384 of faceplate 380. Within the circular raised portion 410 is a spiral groove 430. When backplate 390 is in place, spiral groove 430 forms an evacuated space that allows water to run through it during operation to remove heat from heatsink assembly 280. It is thought that the spiral-shaped channel accommodates all hand piece orientations, and thus is an effective configuration for efficient cooling.

Backplate 390 contains three connectors 440a-440c, which are shown in FIG. 9. When photocosmetic device 100 is fully assembled, connectors 440a-440c provide connections for coolant tube 190a, coolant tube 190b and thermal switch 200, respectively, to allow heatsink assembly 280 to be connected as part of the circulatory system used to cool light source assembly 230. Thus, during operation, water is able to flow from tube 190a, into and through spiral groove 430, and out of heatsink assembly 280 into tube 190b, where the water is returned to coolant reservoir 170. This allows heatsink assembly 280 to cool light source assembly 230 efficiently by transferring additional heat to the approximately 180 cc of water that is contained in the circulatory system. Furthermore, spiral groove 430 provides for efficient heat transfer by providing a relatively long section during which fluid is in contact with heatsink assembly 280.

To assemble heatsink assembly 280, backplate 390 is glued to faceplate 380. Alternatively, backplate 390 could be attached to faceplate 380 by screws or other appropriate means. Other alternative embodiments of heatsink assembly 280 are possible, including alternate configurations of the path that the fluid travels and/or the inclusion of fins or other surfaces to increase the surface area that fluid flows over within the heatsink assembly.

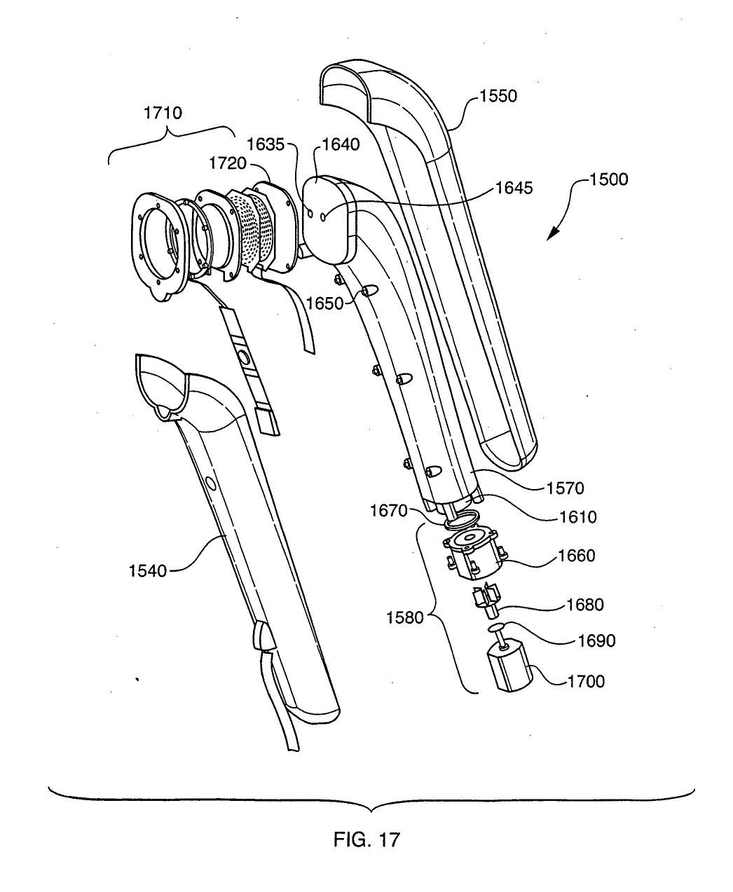

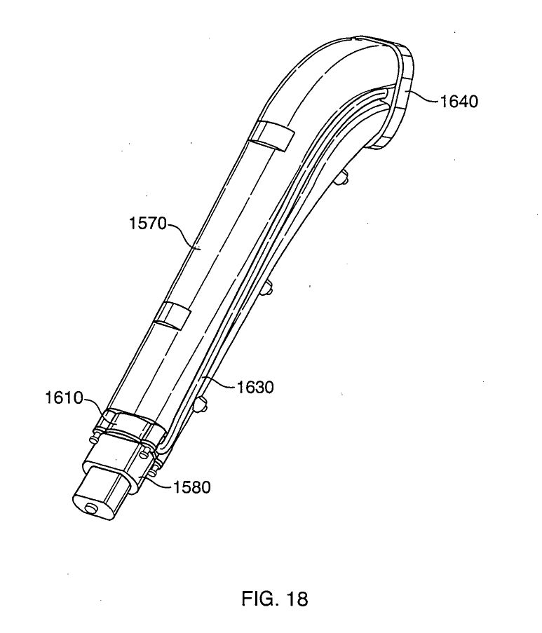

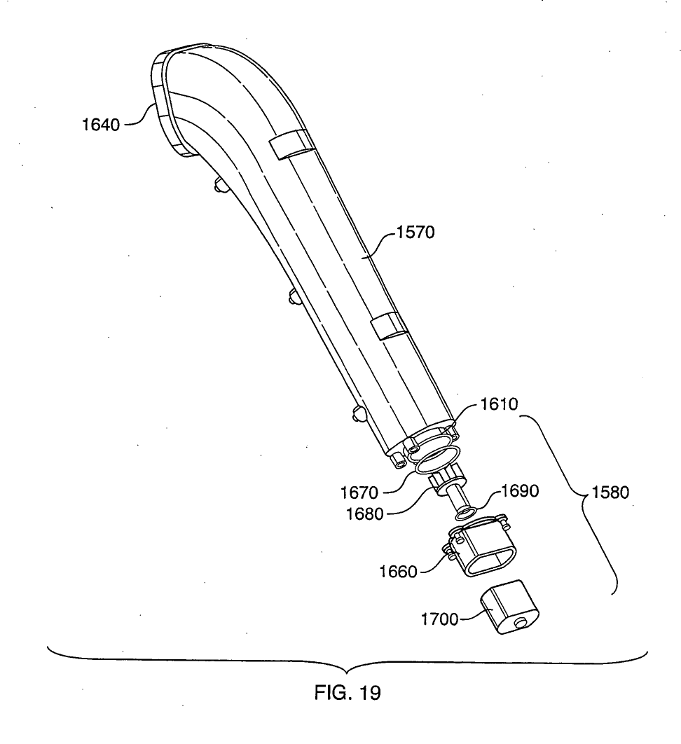

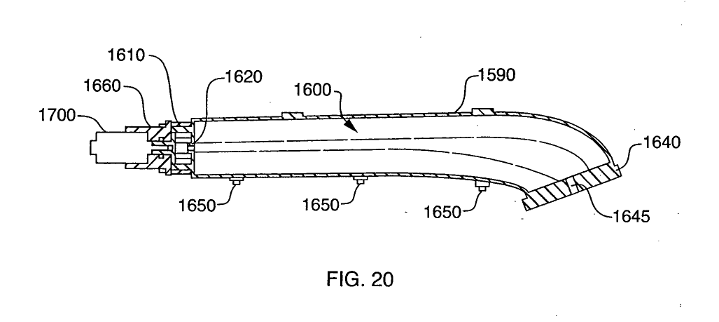

Many other configurations for a circulatory system are possible. One alternate embodiment is shown in FIGS. 17-20. A photocosmetic device 1500, shown in an exploded view in FIG. 17, is similar to photocosmetic device 100, shown in FIG. 1. Photocosmetic device 1500, however, has several differences, including a two-piece design for the housing of photocosmetic device 1500, which is composed of housing sections 1540 and 1550. In comparison, the housing of photocosmetic device 100 is formed by three housing sections 140, 150 and 160, as described above.

Photocosmetic device 1500 also includes a cooling system in which many of the components are integrated into a single reservoir section 1570. The cooling system of photocosmetic device 1500 includes reservoir section 1570 and pump assembly 1580. Reservoir section 1570 includes a housing 1590 that forms reservoir 1600, pump assembly mount 1610, circulatory output 1620, circulatory pipe 1630, interface section 1640, circulatory input 1645 and mounting supports 1650. Pump assembly 1580 includes a motor housing 1660, a motor housing o-ring 1670, an impeller 1680, a motor o-ring 1690, and a DC motor 1700.

When photocosmetic device 1500 is fully assembled, it includes a continuous cooling circuit through which a fluid, in this case water, can circulate to cool light a source assembly 1710 of photocosmetic device 1500. During operation, pump assembly 1580, driven by DC motor 1700, causes coolant to flow through the circulatory system. Coolant preferably flows from reservoir 1600, through circulatory output 1620, where it is pumped by impeller 1680 into circulatory pipe 1630. The coolant travels through the circulatory pipe 1630 and flows into heatsink assembly 1720 via an output opening 1635 in interface section 1640. The output opening 1635 lies at the end of circulatory pipe 1630. The coolant then flows through heatsink assembly 1720, where heat transfers from the heatsink assembly 1720 to the coolant. The coolant then flows back into reservoir 1600 via the input opening 1645 located in the center of the interface section 1640. In photocosmetic device 1500, the heatsink assembly 1720 is a single piece of metal that is secured against the surface of interface section 1640.

In still other embodiments, additional components can be included in the circulatory system to cool a photocosmetic device. For example, a radiator designed to dissipate heat that becomes stored in a coolant reservoir or that either replaces the coolant reservoir or allows for a relatively smaller coolant reservoir, while still accommodating the same amount of heat dissipation and, therefore, treatment time.

Additionally, cooling mechanisms other than circulatory water cooling could be used, for example, compressed gas, paraffin wax with heat fins, or an endothermic chemical reaction. A chemical reactant can be used to enhance the cooling ability of water. For example, NH₄Cl (powder) can be added directly to the coolant (water) to decrease the temperature. This will reduce the heat capacity of water, and, thus, such cooling likely would augment the cooling system as an external cooling source with the NH₄Cl solution separated from the water that is circulated to, e.g., a heatsink near the light source. Alternatively, a suspension of nanoparticles can be used to enhance thermal conductivity of coolant.

Furthermore, other forms of cooling are possible. For example, one advantage of the present embodiment is that it obviates the need for a chiller, which is commonly used to cool photocosmetic devices in the medical setting but which are also expensive and large. However, another possible embodiment could include a chiller either within the handheld photocosmetic device or remotely located and connected by an umbilical cord to the handheld device. Similarly, a heat exchanger could be employed to exchange heat between a first circulatory system and a second circulatory system.

### Electronic Control System

Referring to FIGS. 1-3, photocosmetic device 100 is powered by power supply 215, which provides electrical power to electronic control system 220 via power control switch 210. Power supply 215 can be coupled to photocosmetic device 100 via electrical chord 217. Power supply 215 is an AC adapter that plugs into standard wall outlet and provides direct current to the electrical components of photocosmetic device 100. Electrical chord 217 is preferably lightweight and flexible. Alternatively, electrical chord 217 may be omitted and photocosmetic device 100 can be used in conjunction with a base unit, which is a charging station for a rechargeable power source (e.g., batteries or capacitors) located in an alternative embodiment of photocosmetic device 100. In still other embodiments, the base unit can be eliminated by including a rechargeable power source and an AC adapter in alternate embodiments of a photocosmetic device.

Electronic control system 220 receives information from the components of distal portion 120 over electrical connector 370, for example, information relating to contact of window 240 with the skin via contact sensors 360. Based on the information provided, electronic control system 220 transmits control signals to light source assembly 230 also using electrical connector 370 to control the illumination of the segments 470a-470f of LED module 270. Electronic control system 220 may also receive information from light source assembly 230 via electrical connector 370.

In one embodiment, photocosmetic device 100 is generally safe, even without reliance on the control features that are included. In this embodiment, the energy outputs from photocosmetic device 100 are relatively low such that, even if light from the apparatus was inadvertently shined into a person's eyes, the light should not cause injury to the person's eyes. Furthermore, the person would experience discomfort causing them to look away, blink, or move the light source away from their eyes before any injury could occur. The effect would be similar to looking directly at a light bulb. Similarly, injury to a user's skin should not occur at the energy levels used, even if the recommended exposure intervals are exceeded. Again, to the extent a combination of parameters might result in some injury under some circumstance, user discomfort would occur well before any such injury, resulting in termination of the procedure. Furthermore, the electromagnetic radiation used in embodiments according to the present invention is generally in the range of visible light (although electromagnetic radiation in the UV, near infrared, infrared and radio ranges could also be employed), and electromagnetic radiation such as short-wavelength ultraviolet radiation (<300 nm) that may be carcinogenic or otherwise dangerous can be avoided.

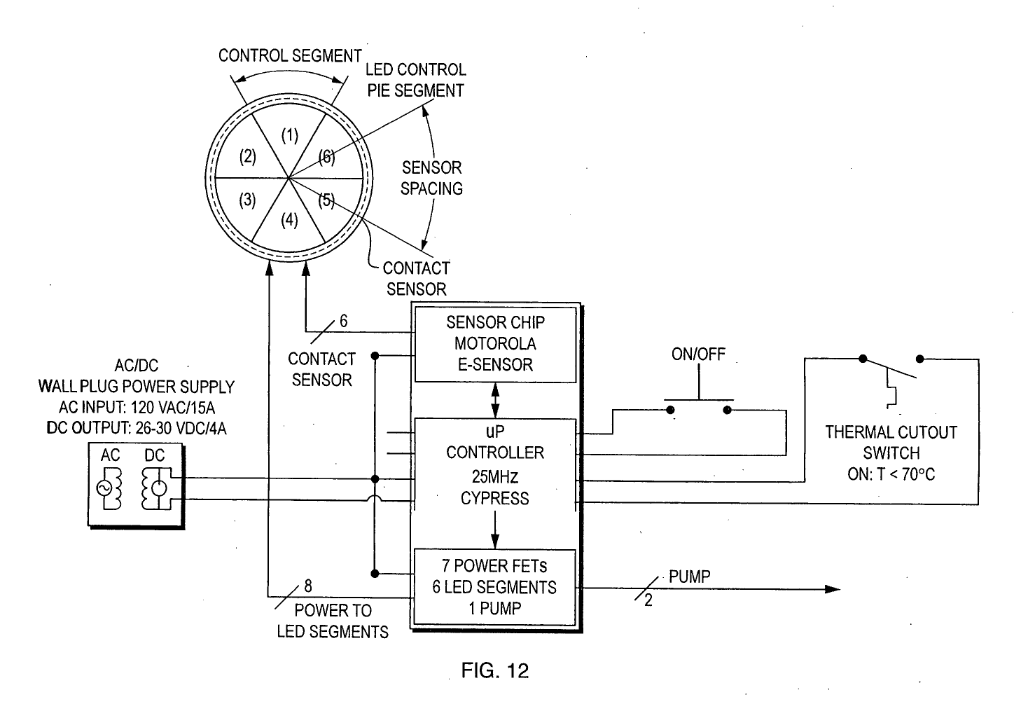

Regardless, although photocosmetic device 100 is generally safe, it contains several additional control features that enhance the safety of the device for the user. For example, photocosmetic device 100 includes standard safety features for an electronic handheld cosmetic device for use by a consumer. Additionally, referring to FIG. 12, photocosmetic device 100 includes additional safety features, such as a control mechanism that prevents use for an extended period by limiting total treatment time, that prevents excessive use by preventing a user from using photocosmetic device 100 again for a preset time period after the a treatment has ended, and that prevents a user from shining the light from photocosmetic device 100 into their eyes or someone else's eyes.

For example, light source assembly 230 may be illuminated only when all or a portion of window 240 is in contact with the tissue to be treated. Furthermore, only those portions of light source assembly 230 that are in contact with the tissue can be illuminated. Thus, for example, LEDs associated with sections of light source assembly 230 that are in contact with the tissue may be illuminated while other LEDs associated with sections of light source assembly 230 that are not in contact are not illuminated.

This is accomplished using contact sensor ring 260, which, as described above, includes a set of six contact sensors 360 located equidistantly around window 240. Each of the six contact sensors 360 are associated with one of the six pie-shaped segments 470a-470f of light source assembly 230. The corresponding LEDs in each segment are activated by the control electronics in response to the sensor output. When a contact sensor 360 detects contact with the skin, an electrical signal is sent to electronic control system 220, which sends a corresponding signal to light source assembly 230 causing the LED dies 530 of the corresponding segment 470a-470f to be illuminated. If multiple contact sensors 360 are pressed, the LED dies 530 of each of the corresponding segments 470a-470f will be illuminated simultaneously. Thus, any combination of the six segments 470a-470f potentially can be illuminated at the same time—from a single segment to all six segments 470a-470f.

In alternative embodiments, the contact sensor can be mechanical, electrical, magnetic, optical or some other form. Furthermore, the sensors can be configured to detect tissue whether window 240 is either in direct contact with or close proximity to the tissue, depending on the application. For example, a sensor could be used in a photocosmetic device having a window or other aperture that is not in direct contact with the tissue during operation, but is designed to operate when in close proximity to the skin. This would allow the device, for example, to inject a lotion or other substance between a window or aperture of the device and the tissue being treated.

In addition to providing a safety feature, contact sensor ring 260 also provides information that can be used by electronic control system 220 to improve the treatment. For example, electronic control system 220 may include a system clock and a timer to control the overall treatment time of a single treatment session. Thus, electronic control system 220 is able to control and alter the overall treatment time depending on the treatment conditions and parameters. Electronic control system 220 can also control the overall power delivered to light source assembly 230, thereby controlling the intensity of the light illuminated from light source assembly 230 at any given point in the treatment.

For example, if during treatment, only one of segments 470a-470f of light source assembly 230 is illuminated, light source assembly 230 will generate only approximately ⅙^{th} of the light energy that would otherwise be generated if all six segments 470a-470f were illuminated. In that case, light source assembly 230 will be generating relatively less heat and be providing relatively less total light to the tissue (although the amount of light per unit area will be the same at that point). If less heat is generated, the water in the cooling system will heat more. slowly, allowing for a longer treatment time. Electronic control system 220 can calculate the rate that energy in the form of light is being provided to the tissue, based on the total time that each of the segments 470a-470f have been illuminated during the treatment session. If less energy is being provided during the course of the treatment, because one or more of the six segments 470a-470f are not illuminated, electronic control system 220 can increase the total treatment time accordingly. This ensures that an adequate amount of light is available to be delivered to the tissue to be treated during a treatment session.

As discussed above, the total possible treatment time for a single treatment using photocosmetic device 100 is approximately ten minutes. If only a portion of the segments 470a-470f are illuminated at various moments during the treatment, electronic control system 220 may extend the treatment time.

Alternatively, if fewer than all six of the segments 470a-470f are illuminated, electronic control system 220 can increase the amount of power available to the illuminated segments 470a-470f, thereby causing relatively more light to be generated by the illuminated sections, which, in turn causes a relative increase in amount of light being delivered per unit area of tissue being treated. This may provide for more effective treatment.

One skilled in the art will appreciate that many variations on the control system of photocosmetic device 100 are possible. Depending on the application and the parameters, total treatment time and light intensity can be varied independently or in combination to effect the desired output. Additionally, an embodiment of a photocosmetic device could include a mode switch that would allow a user to select various modes of operation, including adding additional treatment time or increasing the intensity of the light produced when only some portion of the light sources are illuminated or some combination of the two. Alternatively, the user could choose a higher power but shorter treatment independent of how many segments are illuminated or even if the aperture is not divided into segments.

Furthermore, many alternative configurations of sensors and uses of the device are possible, including one or more velocity sensors that allow the control system of a photocosmetic device to sense the speed at which the user is moving the light source over the tissue. In such an embodiment, when the light source is moving relatively faster, the intensity of the light can be increased by increasing power to the light source to allow the device to continue to provide a more constant amount of light delivered to each unit area of tissue being treated. Similarly, when the velocity of the light source is relatively slower, the intensity of the light can be decreased, and when the light source is not moving for some period of time, but remains in contact with the tissue, the light source can be turned off to prevent damage to the tissue. Velocity sensors can also be used to measure the quality of contact with tissue.

Boost chip 225 provides sufficient power to the electrical components of photocosmetic device 100. Boost chip 225 plays the role of an internal DC-DC converter by transforming the electrical voltage from the power source to ensure that sufficient power is available to illuminate the LED dies 530 of LED module 270.

### Operation of the Photocosmetic Device

In operation, photocosmetic device 100 provides a compact, lightweight hand-held device that a consumer or other user can, for example, use on his/her skin to treat and/or prevent acne. Holding the proximal portion 110, which, among other things, functions as a handle, the user places the micro-abrasive surface 450 of window 240 against the skin. When window 240 is in contact with the skin, the control system in response to the contact sensors illuminates the LED dies 530 of LED module 270. While LED dies 530 are illuminated, the user moves window 240 of photocosmetic device 100 over the surface of the skin, or other tissue to be treated. As window 240 of photocosmetic device 100 moves across the skin, it treats the skin in two ways that work synergistically to improve the health and cosmetic appearance of the skin.

First, micro-abrasive surface 450 removes superficial portions (e.g., dead skin cells and other debris) of the stratum corneum to stimulate desquamation/replacement of the stratum corneum. The human body repeatedly replaces the stratum corneum—replacing the stratum corneum over the course of approximately one month. Removal of old tissue helps to accelerate this renewal process, thereby causing the skin to look better. The micro-abrasive surface 450 is contoured to accentuate the removal of old tissue from the stratum corneum. If there is too little abrasion, the effect will be negligible or non-existent. If there is too much abrasion, the micro-abrasive surface will cut or otherwise damage the tissue. Removal of dead skin can also improve light penetration into the skin.

Second, photocosmetic device 100 treats the skin with light having one or more wavelengths chosen for their therapeutic effect. For the treatment of acne, LED module 270 preferably generates light having a wavelength in the range of approximately 400-430 nm, and preferably centered at 405 nm. Light at those wavelengths has antibacterial properties that assists in the treatment and prevention of acne.

Additionally, light used in conjunction with microdermal abrasion has a therapeutic effect that improves the process of healing wounds on the skin. Although it is not clear that the application of light actually facilitates or speeds the healing process, light appears to provide a beneficial supplemental effect in the healing process. Therefore, it is believed that an embodiment that provides for photo-biomodulation by stimulating the skin with both light and epidermal abrasion will have a beneficial effect on the healing process. Photocosmetic device 100 could be used for such a purpose. As another example, a photocosmetic device having an appropriately contoured micro-abrasive surface and capable of producing light having a wavelength chosen for its anti-inflammatory effects could also be used for such a purpose.

Instead of moving the device across the skin, the device could be used in a “pick and place” mode. In such a mode, the device is placed in contact with or in proximity to the skin /tissue, the LEDs are illuminated for a predetermined pulse width and this is repeated until the entire area to be treated is covered. Such a device may include one or more contact sensors, and the contact sensors alone or the contact sensors and the window 240 may be placed in contact with the skin, and the control system, upon detecting contact, illuminates all or some portion of the LEDs. A micro-abrasive surface may not be as effective in such a device as it would be in a photocosmetic device where the window is moved across the surface of the tissue during operation. To improve the effectiveness of the micro-abrasive surface in a “pick and place” type photocosmetic device, an additional feature, such as a rotating or vibrating window could be included to facilitate microderm abrasion and for other purposes, such as an indication of the completion of the treatment on a particular spot (e.g., communicated to the user by the cessation of movement or vibration).

### User Feedback System

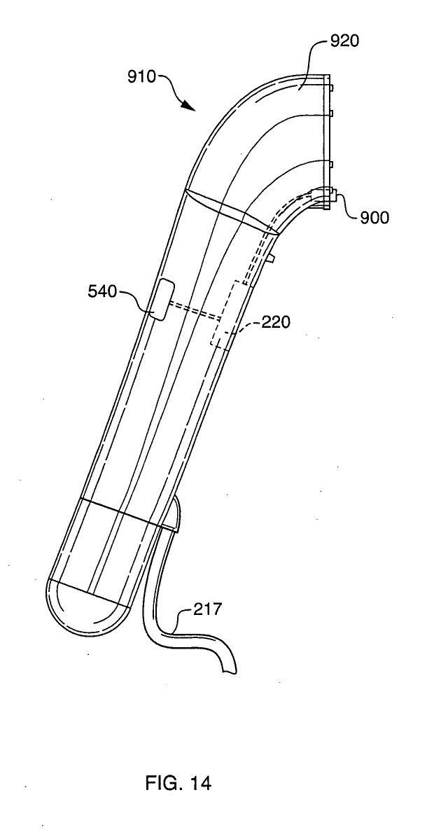

Referring to FIG. 14, an alternative embodiment of a photocosmetic device 910 includes one or more feedback mechanisms. One such feedback mechanism can provide information about the treatment to the consumer. Such a feedback mechanism may include one or more sensors/detectors located in a head 920 of photocosmetic device 910 and an output device 540, which may be located in proximal portion 930. Output device 540 may provide feedback to the user in various forms, including but not limited to visual feedback by illuminating one or more LEDs, mechanical feedback by vibrating the device, sound feedback by emitting one or more tones. The feedback mechanism can be used, for example, to inform the user whether a particular area of tissue contains acne-causing bacteria. In this case, the sensors cause the activation of the output device when acne-causing bacteria is detected to inform the user to continue treating the area. The output device could also be activated, for example, with a different, light, tone or different mechanical feedback, when little to no acne-causing bacteria is detected to indicate that treatment of that area is complete. In other embodiments, additional or different information can be provided to the user, depending on the particular treatment and/or the desired specifications of the device.

Additionally, the same or a different feedback mechanism can provide information to be used by the photocosmetic device 910 to control the operation of the device with or without notifying the user. For example, if the feedback mechanism detects a large amount of acne-causing bacteria, the control system might increase the power to LED module 270 to increase the intensity of the light emitted during treatment of that area to provide more effective treatment. Similarly, if the feedback mechanism detects little or no acne-causing bacteria, the control system might decrease power to the LED module 270 to reduce the intensity of light emitted during treatment of that area to conserve energy and allow for a longer treatment time. If LED module 270 is divided into segments as described above, the device may include one or more feedback mechanisms for each segment and the control system may individually control each segment in response thereto.

In the embodiment shown in FIG. 14, the feedback mechanism includes a sensor 900 that includes a fluorescent sensor used to detect the fluorescence of protoporphrine in acne, which protoporphrins fluoresce after absorbing light in the red and yellow ranges of light. The fluorescence may be a result of the protoporphrins absorbing the treatment light delivered from LED module 270 or the feedback mechanism may include a separate light source for inducing such fluorescence. Areas of increased concentration of bacteria P. Acnes (when treating acne vulgaris) or pigmented oral bacteria (when treating the oral cavity) can be detected and delineated by the fluorescence of proto- and copro-porphyrins produced by bacteria. As treatment progresses, the fluorescent signal decreases.

In other embodiments, a feedback mechanism can be used for detecting, among other things:
a. Changes in skin surface pH caused by bacterial activity.
b. Areas of likely acne lesion formation before the lesion becomes visible. This may be done by detecting changes in skin electrical properties (capacitance) and skin mechanical properties (elasticity).
c. Solar lentigines (pigmentation spots). This may be done by measuring changes in relative melanin and blood content in the local tissue being treated. The same measurement can be used to differentiate between epidermal lesions (to be treated) and moles (treatment to be avoided).
d. Areas of photodamaged skin when performing photorejuvenation. This may be accomplished by measuring the relative change in fluorescence (in particular, collagen fluorescence) of photodamaged vs. non-photodamaged skin.
e. Enamel stains when performing oral treatments. This may be done optically using either elastic scattering or fluorescence. A photodetector and a microchip can be used for detection of reflected and/or fluorescent light from enamel.

A photocosmetic device according to the invention can also treat wrinkles (rhytides) and a sensor to measure the capacitance of the skin can be incorporated into the device, which can be used to determine the relative elasticity of the skin and thereby identify wrinkles, both formed and forming. Such a photocosmetic device could measure either relative changes in capacitance or relative changes in resistance.

A photocosmetic device may also be designed to detect wrinkles, pigmented lesions, acne and other conditions using optical coherence technology (“OCT”). This may be accomplished by pattern recognition in either optical images or skin capacitance images. Such a system may automatically classify, for example, wrinkles and provide additional information to the control electronics that will determine whether and or how to treat the wrinkles. Whether employing OCT, the measurement of electrical parameters, or other detection (or a combination thereof), such devices would have the advantage of controlling/concentrating treatment on the condition itself (e.g., wrinkles, acne, pigmented and vascular lesions, etc.) and may also be used to treat the condition before it fully develops, which may result in better treatment results.

An embodiment of a photocosmetic device could also include a feedback mechanism capable of determining relative changes in pigmentation of the skin to allow treatment of, e.g., age or liver spots or freckles. Such a photocosmetic device could distinguish between pigmentation in the dermis of the skin and pigmentation in the epidermis. During operation, light from one or more LEDs (which may be the treatment source or another light source) penetrates the skin. Some of the light passes only through the epidermis prior to being reflected back to a sensor. Similarly, some of the light passes through both the epidermis and the dermis prior to being reflected back to sensor. An electronic control system can then use the output from the sensors to determine from the reflected light whether the epidermis and dermis contain pigmentation. If the area of tissue being examined includes pigmentation only in the epidermis, the electronic control system may determine that the pigmentation represents a freckle or age spot suitable for treatment. If the area of tissue being examined includes pigmentation in both the dermis and epidermis, the electronic control system may also determine that the tissue contains a mole, tattoo, or dermal lesion that is not suitable for treatment. Such optical pigmentation-sensing system can be implemented using spatially-resolved measurements of diffusely reflected light, possibly in combination with either time- or frequency-resolved detection technique.

It will be clear to one skilled in the art that many alternative embodiments, including different feedback mechanisms with different or additional sensors and light or other energy sources or combinations thereof, are possible. For example, combinations of sensors can be included to measure different physical traits, such as the fluorescence of porphyrins produces by bacteria associated with acne and the skin capacitance associated with wrinkles. Additionally, the placement of sensors can be varied. For example, a photocosmetic device could contain two optical sensors arranged at a right angle or four optical sensors arranged in a square pattern about a light source for treatment to allow the photocosmetic device to sense areas requiring treatment regardless of the direction the user moves the photocosmetic device.

Alternatively, photocosmetic device 100 could include sensors to provide information concerning the rate of movement of window 240 over the user's skin, the existence of acne-causing bacteria and/or skin temperature. In another embodiment, a wheel or sphere may be positioned to make physical contact with the skin, such that the wheel or sphere rotates as the handpiece is moved relative to the skin, thereby allowing the speed of the handpiece to be determined by the control system. Alternatively, a visual indicator (e.g., an LED) or an audio indicator (e.g., a beeper) may be used to inform the user whether the handpiece speed is within the desired range so that the user knows when the device is treating and when it is not. In some embodiments, multiple indicators (e.g., LEDs having different colors, or different sound indicators) may be used to provide information to the user.

It should be understood that other methods of speed measurement are with the scope of this aspect of the invention. For example, electromagnetic apparatuses that measure handpiece speed by recording the time dependence of electrical (capacitance and resistance)/magnetic properties of the skin as the handpiece is moved relative the skin. Alternatively, the frequency spectrum or amplitude of sound emitted while an object is dragged across the skin surface can be measured and the resulting information used to calculate speed because the acoustic spectrum is dependent on speed. Another alternative is to use thermal sensors to measure handpiece speed, by using two sensors separated by a distance along the direction in which the handpiece is moved along the skin (e.g., one before the optical system and one after). In such embodiments, a first sensor monitors the temperature of untreated skin, which is independent of handpiece speed, and a second sensor monitors the post-irradiation skin temperature; the slower the handpiece speed, the higher the fluence delivered to a given area of the skin, which results in a higher skin temperature measured by the second detector. Therefore, the speed can be calculated based on the temperature difference between the two sensors.

In any of the above embodiments, a speed sensor may be used in conjunction with a contact sensor (e.g., a contact sensor ring 260 as described herein). In one embodiment of a handpiece, both contact and speed are determined by the same component. For example, an optical-mouse-type sensor such as is used on a conventional computer optical mouse may be used to determine both contact and speed. In such a system, a CCD (or CMOS) array sensor is used to continuously image the skin surface. By tracking the speed of a particular set of skin features as described above, the handpiece speed can be measured and because the strength of the optical signal received by the array sensor increases upon contact with the skin, contact can be determined by monitoring signal strength. Additionally, an optical sensor such as a CMOS device may be used to detect and measure skin pigmentation level or skin type based on the light that is reflected back from the skin; a treatment may be varied according to pigmentation level or skin type.

In some embodiments of the present invention, a motion sensor is used in conjunction with a feedback loop or look-up table to control the radiation source output. For example, the emitted laser power can be increased in proportion to the handpiece speed according to a lookup table. In this way, a fixed skin temperature can be maintained at a selected depth (i.e., by maintaining a constant flux at the skin surface) despite the fact that a handpiece is moved at a range of handpiece speeds. The power used to achieve a given skin temperature at a specified depth is described in greater detail in U.S. patent application Ser. No. 09/634,981, which is incorporated herein by reference. Alternatively, the post-treatment skin temperature may be monitored, and a feedback loop used to maintain substantially constant fluence at the skin surface by varying the treatment light source output power. Skin temperature can be monitored by using either conventional thermal sensors or a non-contact mid-infrared optical sensor. The above motion sensors are exemplary; motion sensing can be achieved by other means such as sound (e.g., using Doppler information).

### Optical Attachments for Use with a Photocosmetic Device

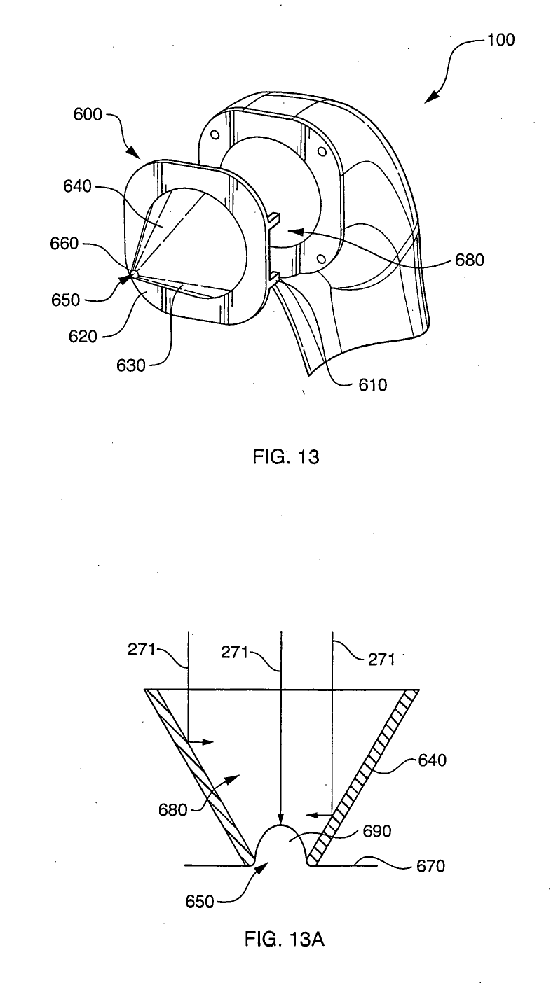

Photocosmetic device 100 optionally may include attachments to assist the user in performing various treatments or aspects of treatments. For example, an attachment may be used to treat tissue in hard-to-reach areas such as around the nose. Photocosmetic devices that use attachments or other mechanisms to control or change the aperture can be referred to as having “adaptive apertures.” Referring to FIG. 13, an attachment 600 for photocosmetic device 100 is shown. Attachment 600 attaches to the distal portion 120 of photocosmetic device 100 by clips 610. Four clips are symmetrically arranged with two clips on each of two opposite sides of attachment 610. Attachment 600 includes a frame 620 and an aperture 630. Aperture 630 is cone-shaped and includes an opaque cone section 640 and an opening 650. The surface of opaque section 640 that faces photocosmetic device 100 when attachment 600 is attached is coated with a reflective material. Opening 650 allows light to pass and may be an actual opening or it may have a window across it which may be made of the same material as window 240.

When attachment 600 is attached to photocosmetic device 100, aperture 630 covers window 240 such that, when light source assembly 230 is illuminated, essentially all of the light passes through aperture 630. During operation, attachment 600 allows the user to concentrate the light onto a smaller area of tissue to be treated. By way of example, a user may attach attachment 600 to photocosmetic device 100 to treat a specific small affected area, such as an individual pimple, individual wrinkles or other conditions (e.g., small blood vessel or pigmented lesion) in an area that difficult to reach such as around the nose.

The user may place the edge 660 of opening 650 against the skin. Such contact would allow frame 620 of attachment 600 to engage a pressure sensitive switch in photocosmetic device 100 via the clips 610. When attachment 600 is pressed against the tissue, it closes the switch, which completes a circuit causing the contact sensors 360 to appear to be engaged. Thus, electronic control system 220 causes all six segments 470a-470f to be simultaneously illuminated. Alternatively, attachment 600 could include a wire that runs around the surface of frame 620 that faces the contact sensors 360 that forms a completed circuit when attachment 600 is attached to photocosmetic device 100 and the attachment 600 is pressed against the tissue, which would cause sensors 360 to detect an electronic field and allow each of the six segments 470a-470f to be illuminated.

As shown in FIG. 13A, the light, represented by arrows 271, generated by LED module 270 either passes directly through opening 650 or is reflected by the interior reflective surface of opaque cone section 640. Because light source assembly 230 also includes a optical reflector 490, most of the light will continue to be reflected within a space 680 bounded by aperture 630 and optical reflector 490 until it passes into the tissue 670 that is being treated or is absorbed by a surface of photocosmetic device 100. Relatively more light will be concentrated onto tissue 670, if material having relatively higher reflectivity is used and if relatively more of the surface within space 680 is coated with reflective material.

Opening 650 shown in FIG. 13A is not covered by a window and in operation tissue 670 is slightly distended within cone 640 when rim 660 is pressed against tissue 670. A portion 690 of tissue 670, which may, for example, be a pimple symptomatic of acne, is located within space 680. This allows light 271 to strike the top of tissue 690 directly from light source assembly 230 and to strike the side of tissue 690 indirectly as light 271 is reflected by the interior surface of opaque cone section 640. Allowing the pimple represented by portion 690 to be bathed in light from both the top and sides is believed to improve the therapeutic effect of the light treatment and more effectively reduce or eliminate the pimples treated.

In addition to treating pimples, attachment 600 can also be used for other purposes. For example, attachment 600 can be used to treat areas of tissue that are difficult to treat using the larger surface of window 240, such as the crevice between the cheek and the nostrils. Attachment 600 can be used to treat along an individual wrinkle or to provide carefully directed treatment in sensitive areas, such as around the eyes.

Many different embodiments of attachment 600 are possible. For example, alternative embodiments of a photocosmetic device can include electrical contacts or other mechanisms that inform the electrical control system when an attachment is connected. That would allow the electrical control system, for example, to change the mode of operation by increasing or decreasing power to the light source or only illuminating a portion of the light sources when more than one light source is available (e.g., array of LEDs), changing the pulse-width and power of the output from the light source (e.g., treating the tissue with a higher power pulse of light for a shorter duration of time or lower power with longer duration), altering the treatment time, using contact sensors placed on the end of the attachment and ignoring the information from the contact sensors on the window, etc. That would also allow the electronic control system to distinguish between various adapters to be used for various purposes with the device.

The size, shape, dimensions and materials of attachment 600 also can be varied. By way of example, an attachment could be shaped as a pyramid. Similarly, the interior reflective surface of the attachment could conform to a logarithmic curve to more directly reflect light onto the tissue and reduce the amount of light that is reflected back toward the photocosmetic device. As another example, the attachment may be a simple, flat mask that allows light to pass only from a portion of the window 240. In addition, the opening need not be centered on window 240 but can be off to one side. Similarly, the opening can be varied in size and shape and may also have focusing or other optics across the front of or behind the opening. Several attachments may be made available for connection to the photocosmetic device to serve different functions, and each member of a family might have their own attachment in the same manner that each family member has their own toothbrush head for connection to a common electric toothbrush base. Instead of concentrating the light onto a smaller area than window 240, an attachment could be provided to deliver the light onto a larger treatment area. The aperture of the device also can have different shapes, for example, to effectively accommodate various tissue types, tissue contours, and treatments.

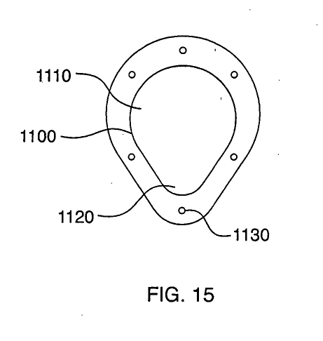

Other embodiments can be used to facilitate the treatment of areas that are difficult to reach with light emitted from a relatively larger surface. For example, as shown in FIG. 15, a window 1100 of a photocosmetic device can be shaped as a teardrop having a broader surface portion 1110 and a narrower surface portion 1120. The user could use the entire surface of window 1100 to treat relatively flat areas of tissue, and, alternatively, could use the narrower surface portion 1120 to treat areas of tissue that are difficult to treat with a larger surface. When the user uses only the narrower surface portion 1120 of window 1100 to treat tissue, only the LEDs associated with the narrower surface portion may be illuminated. For example, a contact sensor 1130 associated with narrower surface portion 1120 may be in contact with or close proximity with the tissue to be treated using narrower surface portion 1120 while the contact sensors associated with broader surface portion 1110 are not engaged. The control system may then use this contact information to illuminate only the LEDs associated with narrower surface portion 1120. This configuration may eliminate the need for an add-on component such as attachment 600.

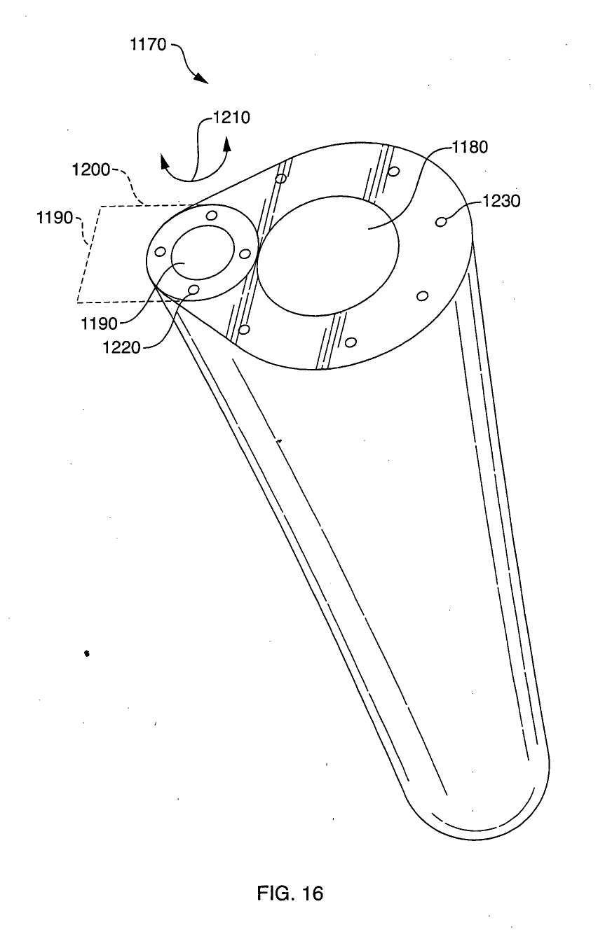

Referring to FIG. 16, in still another embodiment, a photocosmetic device 1170 can have two (or more) independent apertures: a large window 1180 and small window 1190. Optionally, the windows may be movable relative to one another. Small window 1190 may be located at the end of an arm 1200 that swings to and from an extended position as show by arrow 1210. When fully extended, arm 1200 locks in place. During treatment with arm 1200 extended, one or more contact sensors 1220 associated with small window are placed in contact with or in close proximity to the tissue to be treated, while contact sensors 1230 associated with large window 1180 are not engaged. Thus, only the light source (e.g., LEDs) associated with small window 1190 will be illuminated when the photocosmetic device is used in this manner, and the LEDs associated with large window 1180 will not be illuminated. Furthermore, as discussed above in relation to photocosmetic device 100, the control system of photocosmetic device 1170 can determine that only a relatively smaller portion of the available window area is being utilized, and can increase the power to the LEDs associated with either small window 1190 or when using the larger window 1180 (or when using both the smaller and larger windows simultaneously). That will result in more light being produced by those LEDs and, thus, may increase the efficacy of certain treatments.

Optionally, a tip reflector may be added around the one or more apertures to redirect light scattered out of the skin back into the skin (described above as photon recycling). For wavelengths in the near-IR, between 40% and 80% of light incident on the skin surface is scattered out of the skin; as one of ordinary skill would understand the amount of scattering is partially dependant on skin pigmentation. By redirecting light scattered out of the skin back toward the skin using a tip reflector, the effective fluence provided a photocosmetic device can be increased by more than a factor of two. Tip reflectors may have a copper, gold or silver coating to reflect light back toward the skin.

A reflective coating may be applied to any non-transmissive surfaces of the device that are exposed to the reflected/scattered light from the skin. As one of ordinary skill in the art would understand, the location and efficacy of these surfaces is dependent on the chosen focusing geometry and placement of the light source(s).

### Additional Embodiments

Given the detailed description above, it is clear that numerous alternative embodiments are possible. For example, dimensions, attachments, wavelengths of light, treatment times, modes of operation and most other parameters can be varied depending on the desired treatment and the method of treatment.

For example, light sources with mechanisms for coupling light into the skin can be mounted in or to any hand piece that can be applied to the skin, for example any type of brush, including a shower brush or a facial cleansing brush, massager, or roller. See, for example, U.S. application entitled, Methods And Apparatus For Delivering Low Power Optical Treatments, U.S. application Ser. No. 10/702,104 filed Nov. 4, 2003, Publication No. US 2004/0147984 A1, published Jul. 29, 2004, which is incorporated herein by reference in its entirety. In addition, the light sources can be coupled into a shower-head, a massager, a skin cleaning device, etc. The light sources can be mounted in an attachment that may be clipped, fastened with Velcro or otherwise affixed/retrofitted to an existing product or the light sources can be integrated into a new product.

In another alternative embodiment, a photocosmetic device can be attached to a person such that the person need not hold the device during operation, e.g., by tape, a strap or a cuff. Such a device could provide light to an area of tissue to, e.g., kill or prevent bacteria from growing in a wound, decrease or eliminate inflammation in the tissue, or provide other therapeutic effects. Such a device could take advantage of the heat produced by the light source by, e.g., including a cuff as part of the cooling system and circulating water through the cuff that has been heated by the heat produced by the light source. Such a device could provide additional heating of tissue similar to a heating pad.

Alternatively, a device could be used to apply “cold” to the tissue, by, for example, including a compartment or container for inserting ice or a re-freezable packet that would assist in cooling both the device and the tissue to be treated. Such a device could use the ice or other cooling mechanism to both cool the tissue to be treated as well as cool any fluid circulating in the coolant system of the device, thereby providing for a longer treatment time, a relatively smaller device requiring less coolant during operation, or both. Such a device could include a container that is removable, reusable and/or refillable. It could also include disposable containers. The containers could be filled with various fluids, mixtures of fluids or mixtures of fluids and solid particles, depending on the application.

Although a closed circulatory system has been described, other configurations are possible, including an open cooling circuit in which a source or fluid supply, such as a refillable container, is inserted into the device to provide a fluid, such as water, to cool the device.

An embodiment of the invention may also be in the form of a face-mask or in a shape to conform to other portions of a user's body to be treated, the skin-facing side of such applicator having an aperture or apertures with exterior surfaces that are smooth, contoured or flat or that utilize projections, water jets or bristles to deliver the radiation. While such an apparatus could be moved over the user's skin, to the extent it is stationary, it would not need to provide the abrading or cleaning action of the preferred embodiments.

The head of an alternative embodiment could also have openings through which a substance such as a lotion, drug or topical substance is dispensed to the skin before, during or after treatment. Such lotion, drug, topical substance or the like could, for example, contain light activated compounds to facilitate certain treatments. The lotion could also be applied prior to the treatment, either in addition to, or instead of, applying during treatment. Such a device could be used in conjunction with an antiperspirant or deodorant lotion to enhance the interaction between the lotion and the sweat glands via photothermal or photochemical mechanisms. The lotion, drug or topical substance can contain compounds with different benefits for the skin and human health, such as skin cleaning, moisturizing, collagen production, etc.

### Use of Light of Different Wavelengths in a Photocosmetic Device

Additionally, in alternative embodiments, depending on the desired treatment, different wavelengths of light will enhance the effect. For example, when treating acne, a wavelength band from 290 nm to 700 nm is generally acceptable with the wavelength band of 400-430 nm being preferred as described above. For the stimulation of collagen, the target area for this treatment is generally the papillary dermis at a depth of approximately 0.1 mm to 0.5 mm into the skin, and since water in tissue is the primary chromophore for this treatment, the wavelength from the radiation source should be in a range highly absorbed by water or lipids or proteins so that few photons pass beyond the papillary dermis. A wavelength band from 900 nm to 20000 nm meets these criteria. For sebaceous gland treatment, the wavelength can be in the range 900-1850 nm, preferable around peaks of lipid absorption as 915 nm, 1208 nm, and 1715 nm. Hair growth management can be achieved by acting on the hair follicle matrix to accelerate transitions or otherwise control the growth state of the hair, thereby accelerating or retarding hair growth, depending on the applied energy and other factors, preferable wavelengths are in the range of 600-1200 nm.

In alternative embodiments, the light source may generate outputs at a single wavelength or may generate outputs over a selected range of wavelengths or one or more separate bands of wavelengths. Light having wavelengths in other ranges can be employed either alone, or in conjunction with other ranges, such as the 400-430 nm to take advantage of the properties of light in various ranges. For example, light having a wavelength in the range of 480-510 nm is known to have anti-bacterial properties, but is also known to be relatively less effective in killing bacteria than light having wavelengths in the range of 400-430 nm. However, light having a wavelength in the range of 480-510 nm also is known to penetrate relatively deeper into the porphyrins of the skin than light in the range of 400-430 nm.

Similarly, light having a wavelength in the range of 550-600 nm is known to have anti-inflammatory effects. Thus, light at these wavelengths can be used alone in a device designed to reduce and/or relieve inflammation and swelling of tissue (e.g., inflammation associated with acne). Furthermore, light at these wavelengths can be used in combination with the light having the wavelengths discussed above in a device designed to take advantage of the characteristics and effects of each range of wavelengths selected.

In embodiments of a photocosmetic device capable of treating tissue with light of multiple wavelengths, multiple light sources could be used in a single device, to provide light at the various desired wavelengths, or one or more broad band sources could be used with appropriate filtering. Where a radiation source array is employed, each of several sources may operate at selected different wavelengths or wavelength bands (or may be filtered to provide different bands), where the wavelength(s) and/or wavelength band(s) provided depend on the condition being treated and the treatment protocol being employed. Similarly, one or more broadband sources could be used. For a broadband source, filtering may be required to limit the output to desired wavelength bands. An LED module could also be used in which LED dies that emit light at two or more different wavelengths are mounted on a single substrate and electrically connected to all the various dies to be controlled in a manner suitable for the treatment for which the device is designed, e.g., controlling some or all of the LED dies at one wavelength independently or in combination with LED dies that emit light at other wavelengths.

Employing sources at different wavelengths may permit concurrent treatment for a condition at different depths in the skin, or may even permit two or more conditions to be treated during a single treatment or in multiple treatments by selecting a different mode of operation of a photocosmetic device. Examples of wavelength ranges for various treatments are provided in the table below.

TABLE 3
Uses of Light of Various Wavelengths In Photocosmetic Procedures

| Treatment condition or application | Wavelength of Light, nm |
|---|---|
| Anti-aging | 400-2700 |
| Superficial vascular | 290-600 |
| | 1300-2700 |
| Deep vascular | 500-1300 |
| Pigmented lesion, de pigmentation | 290-1300 |
| Skin texture, stretch mark, scar, porous | 290-2700 |
| Deep wrinkle, elasticity | 500-1350 |
| Skin lifting | 600-1350 |
| Acne | 290-700, 900-1850 |
| Psoriasis | 290-600 |
| Hair growth control, | 400-1350 |
| PFB | 300-400, 450-1200 |
| Cellulite | 600-1350 |
| Skin cleaning | 290-700 |
| Odor | 290-1350 |
| Oiliness | 290-700, 900-1850 |
| Lotion delivery into the skin | 1200-20000 |
| Color lotion delivery into the skin | Spectrum of absorption of |
| | color center and |
| | 1200-20000 |
| Lotion with PDT effect on skin | Spectrum of absorption of |
| condition including anti cancer effect | photo sensitizer |
| ALA lotion with PDT effect on skin | 290-700 |
| condition including anti cancer effect | |
| Pain relief | 500-1350 |
| Muscular, joint treatment | 600-1350 |
| Blood, lymph, immune system | 290-1350 |
| Direct singlet oxygen generation | 1260-1280 |

In other alternative embodiments, the size and shape of the head of a photocosmetic device can be varied depending on the tissue that the photocosmetic device is designed to treat. For example, the head could be larger to treat the body and smaller to treat the face. Similarly, the size, shape and number of the aperture(s) of such a device can be varied. Also, a set of replaceable heads could be used - each head having various designs to serve different functions for a specific treatment or allowing one device to be used for multiple treatments. Similarly, only a portion of the head could be replaceable, such as the face of the head with the aperture through which the light is emitted, without replacing the light source, to avoid the additional cost of having multiple light sources.

A larger photocosmetic device may, for example, be used on the body during a shower or bath. In that situation, the water could also act as a waveguide for the light being delivered to the user's skin. A smaller photocosmetic device can be used to provide more targeted treatment to smaller areas of tissue or to treat difficult-to-reach areas of tissue, e.g., in the mouth or around the nose.

To this point, embodiments of the invention have been described predominately with respect to photocosmetic treatments for the skin. However, other tissues can be treated using embodiments according to the present invention, including finger and toenails, teeth, gums, other tissues in the oral cavity, or internal tissues, including but not limited to the uterine cavity, prostate, etc.

In another embodiment, the devices described herein can be adapted such radiation is emitted primarily by light sources positioned over and/or passing over areas detected for treatment. For example, as the device that travels over the skin, a controller turns on only certain light sources that correspond to areas detected for treatment. For example, if passing over the skin a small pigmented lesion is detected, only a portion of the LEDs that will pass over that lesion could be illuminated to avoid wasting energy by applying light to tissue that doesn't need treatment.

### A Photocosmetic Device for Treatment of Tissues in the Oral Cavity

There are several conditions that may be treated using embodiments according to aspects of the present invention designed for use in the oral cavity. For example, embodiments according to the present invention can treat conditions within the mouth such as those caused by excessive plaque buildup or bacteria in the mouth. Such methods are described in greater detail in both U.S. application Ser. No. 10/776,667, entitled “Dental Phototherapy Methods And Compositions, filed Feb. 10, 2004 and International Publ. No. WO 2004/084752 A2, entitled “Light Emitting Oral Appliance and Methods of Use,” published Oct. 7, 2004, which are incorporated herein by reference.

Additionally, by using devices according to aspects of the present invention to treat tissues in the mouth, certain conditions, which had in the past been treated from outside the oral cavity, may be treated by employing an optical radiation source from within the oral cavity. Among these conditions are acne and wrinkles around the lips. For example, instead of treating acne, for example, on the cheek, by radiating the external surface of the affected skin, oral appliances can radiate the cheek from within the oral cavity out toward the target tissue. This is advantageous because the tissue within the oral cavity is easier to penetrate than the epidermis of the external skin due to absence of melanin in the tissue walls of the oral cavity and lower scattering in the mucosa tissue. As a result, optical energy more easily penetrates tissue to provide the same treatment at a lower level of energy and reduce the risk of tissue damage or improved treatment at the same level of energy. A preferable range of wavelength for this type of treatment is in the range of about 280 nm to 1400 nm and even more preferably in the range of about 590 nm -1300 nm.

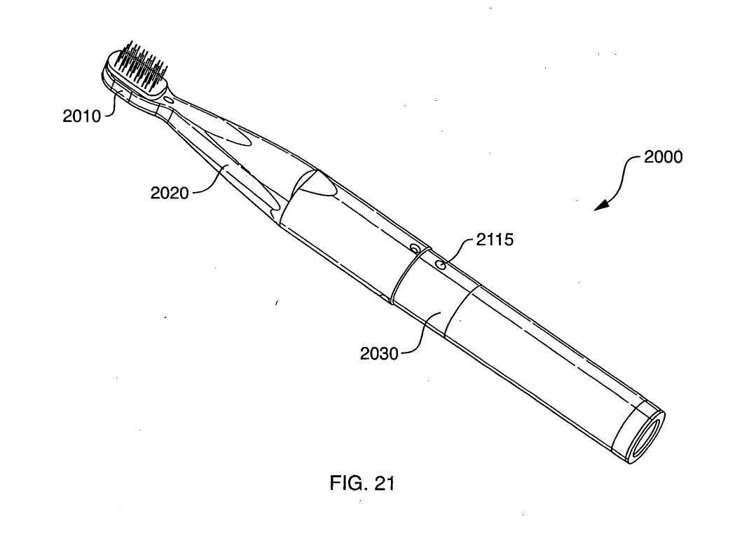

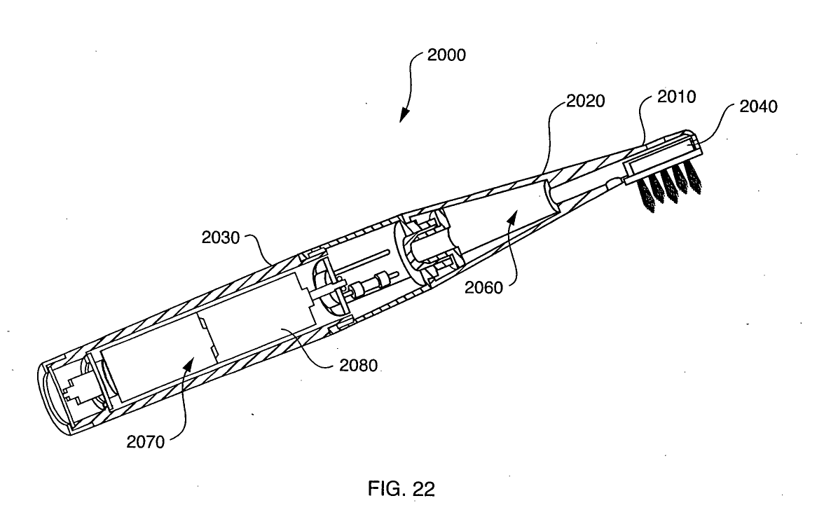

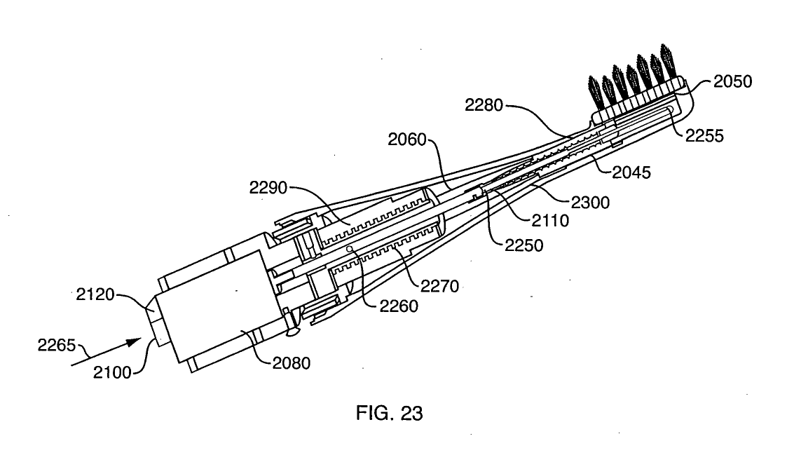

Referring to FIGS. 21-23, another embodiment of a photocosmetic device 2000 is shown. Photocosmetic device 2000 is a toothbrush used to treat tissue in a user's mouth, such as teeth, gums, and other tissue. Photocosmetic device 2000 includes a head portion 2010, a neck portion 2020 and a handle portion 2030.

Head portion 2010 may be a removable toothbrush head to allow it to be replaced periodically. Alternatively, head portion 2010 would not be removable and photocosmetic device 2000 could have a unibody design. Head portion 2010 includes a heatsink 2040 and a light source assembly 2050 for treating tissues in the mouth.

Neck portion 2020 includes a coolant reservoir 2060 that, during operation, is filled with, for example, water, which is circulated through head portion 2010 to cool light source assembly 2050 by removing excess heat from heatsink 2040.

Handle portion 2030 includes a compartment 2070 where batteries are installed to power photocosmetic device 2000, and additionally includes a motor 2080, a PCM heat capacitor 2090, a booster chip 2100, a helical pump 2110, a power switch 2115 and electronic control system 2120. Electronic control system 2120 controls the illumination of light source assembly 2050 and may provide feedback to the user through one or more feedback mechanisms as described above, e.g., to identify for the user the presence of bacteria requiring additional treatment. Helical pump 2110 circulates fluid, such as water, that is used as a coolant for cooling the light source assembly 2050 of photocosmetic device 2000.

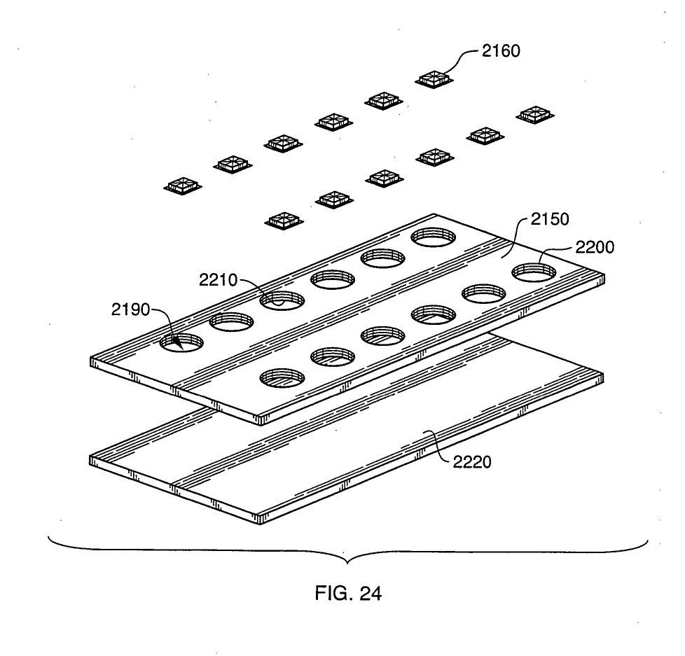

Light source assembly 2050 is shown in greater detail in FIGS. 24 through 26. Light source assembly 2050 includes a bristle assembly 2130 mounted on an LED module 2140 that has an optical reflector 2150 capable of reflecting 95% or more of the light emitted from LED dies 2160 of LED module 2140.

Bristle assembly 2130 includes twelve stands of transparent light-transmitting optical bristles 2170 that are attached to a mounting platform 2180. Mounting platform 2180 includes a set of holes (not shown) to accommodate the bristles 2170, when the bristles 2170 are mounted.

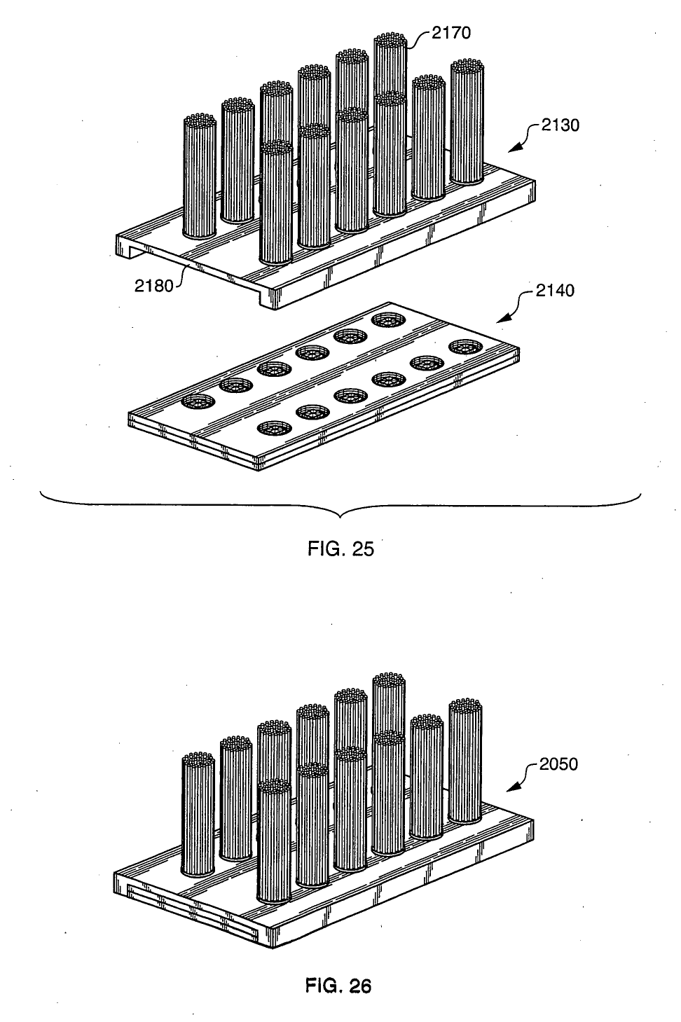

Optical reflector 2150 is a photorecycling mirror that contains an array of holes 2190. Each hole 2190 is funnel-shaped having a cone section 2200 and a tube section 2210. Each of the holes 2190 correspond to one of the individual LED die 2160 that are mounted on a substrate 2220. Thus, when assembled, as shown in FIG. 25, each hole 2190 accommodates one LED die 2160. Optical reflector 2150 is made from OHFC copper that has been plated with silver, but can be of any material provided it is highly reflective preferably on all surfaces that make contact with light. The reflective surfaces of optical reflector 2150 are provided to more efficiently reflect additional light generated by the LED module 2140 through the bristles 2170 and onto the tissue to be treated.

The assembly process for LED module 2140 is illustrated with reference to FIG. 24. First, optical reflector 2150 is attached to substrate 2220, which is a patterned metallized ceramic. Second, the individual LED dies 2160 are mounted to substrate 2220 through the holes 2190 in optical reflector 2150. The material used to attach LED dies 2160 to substrate 2220 should be suitable for minimizing chip thermal resistance. A suitable solder could be eutectic gold tin and this could be pre-deposited on the die at the manufacturer. Third, the LED dies 2160 are Au wire bonded to provide electrical connections. Finally, the LED dies 2160 are encapsulated with the appropriate index matching optical gel (coupling medium) and the output optics is added to complete the encapsulation. Various optical coupling media can be used for the purpose (e.g., NyoGels by Nye Optical).

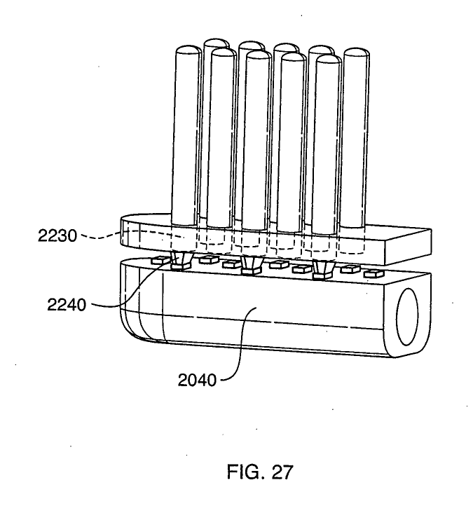

The light-transmitting bristles 2170 are mounted within mounting platform 2180 to form bristle assembly 2130. Bristle assembly 2130 is then glued to the top surface of LED module 2140 such that each individual stand of bristles 2170 are positioned directly adjacent to each of the LED dies 2160 to allow light emitted from the LED die to pass through the light-transmitting optical bristles 2170. As illustrated in FIG. 27, a proximal end 2230 of each stand of bristles 2170 is coupled to a corresponding LED die 2160 by an optical coupler 2240, which is made of a suitable optical material, to more efficiently transfer light from the LED die 2160 to the bristles 2170.

As shown in FIGS. 21 through 23, during operation, the user turns on photocosmetic device 2000 using power switch 2115. This closes an electronic circuit that causes power to be supplied from batteries (not shown). Thus, as electronic control system 2120 operates, light source assembly 2050 is illuminated, and motor 2080 operates and begins to turn helical pump 2110. Helical pump 2110 pumps coolant, here water, by turning a thread 2245, which is located on the external surface of a central shaft 2250 of helical pump 2110 and extends from the central shaft 2250 to approximately the inner cylindrical surface 2280 of neck portion 2020. The turning movement of thread 2245 forces water through the cooling system, which is a continuous circuit.

Helical pump 2110 causes water to flow from coolant reservoir 2060 and through heatsink 2040 of head portion 2010. During operation, heat produced by light source assembly 2050 conducts through heatsink 2040. The excess heat is transferred from heatsink 2040 to the water circulating through heatsink 2040. The heated water then flows into an open end 2255 of central shaft 2250, which forms a hollow tube running along a longitudinal axis 2265 from head portion 2010, through neck portion 2020, and to handle portion 2130. The heated water flows through central shaft 2250 and is expelled from the interior of central shaft 2250 through holes 2260 that are located adjacent to the heat capacitor 2090. At this point, the heated water reverses direction, and flows along fins 2270 of heat capacitor 2090, to more efficiently transfer heat from the water to the heat capacitor 2090. The water then flows around the exterior of central shaft 2250 back into the coolant reservoir 2060 of neck portion 2020.

To prevent water from flowing out of the cooling system, the cooling system is sealed appropriately, including with a seal 2290 between heat capacitor 2090 and motor 2080. Because head portion 2010 is removable, the junction 2300 between head portion 2010 and neck portion 2020 must also be sealed to prevent photocosmetic device 2000 from leaking. This is accomplished by designing a close fit between the head and neck portions 2010 and 2020 that snap together and effectively seal the cooling system.

The user places the head portion 2010 in the oral cavity and brushes the tissue to be treated with the bristles 2170. Light radiates from the bristles to the tissue being treated. For example, light can be used to treat plaque deposits on the teeth and remove bacteria from teeth and gums.

The specifications of photocosmetic device 2000 are shown in the table below, along with an alternative low-power embodiment of photocosmetic device 2000. The low power embodiment has the advantage of using less power. Thus, a circulatory cooling system is not required. Instead, a heatsink is provided that allows heat generated by a light source to be stored in the head, neck and handle portions of the photocosmetic device and directly radiated from the photocosmetic device to the surrounding air, the user's hand on the hand piece and/or the user's oral tissue.

TABLE 4
Specifications For Two Embodiments Of A Photocosmetic
Device For Treating Tissue In The Oral Cavity

| Parameters | Low power version | High power version |
|---|---|---|
| Power, mW | 10-50 | 250-1000 |
| One wavelength version, nm | 405, 500, 630, | 405, 500, 630, |
| | 660, 1450 | 660, 1450 |
| Dual wavelength | 405/630 (70/30%) | 405/630 (50/50%), |
| version, nm | | 405/1450 (50/50%) |
| Treatment time, min | 3 | 3 |
| Power supply | Battery | Battery |
| Weight, lb | 0.35 Lbs | 0.5 lbs |
| Bristle | Transparent with | Transparent with |
| | more than | more than |
| | 75% power | 25% power |
| Photon recycling | Yes | Yes |
| Directional | Mono | Mono |

In another embodiment, a photocosmetic device for treating tissues in the oral cavity can include a feedback mechanism, including a sensor that provides information about treatment results, such as the existence of problematic areas to be treated by the user as well as an indication that treatment is complete. The feedback sensor could be a fluorescent sensor used to detect the fluorescence of bacteria that, for example, causes bad breath or other conditions of the tissue in the oral cavity. The sensor can detect and delineate pigmented oral bacteria by the fluorescence of proto- and copro-porphyrins produced by bacteria. As treatment progresses, the fluorescent signal will decrease and the feedback mechanism can include an output device, as described above, to indicate to the user when treatment is completed or areas that the user needs to continue treating.

The user can direct light from the bristles to any tissue within the oral cavity, for example, teeth, gums, tongue, cheek, lips and/or throat. In another embodiment of the invention, the applicator may not include bristles but instead include a flat surface, or surface with bumps or protrusions or some other surface for applying light to the tissue. The applicator can be pressed up against the oral tissue such that it contacts the tissue at or near a target area. The applicator can be mechanically agitated in order to treat the subsurface organs without moving the applicator from the contact area. For example, an applicator can be pressed up against a user's cheek, such that the applicator contacts the user's cheek at a contact area. The applicator can be massaged into the user's cheek to treat the user's teeth or underlying glands or organs while the physical contact point remains unchanged. The head of such an applicator can contain a contact window composed of a transparent, heat transmitting material. The contact window can be adapted to be removable so that it can be replaced by the user.

In other embodiments, optical radiation can be directed in multiple directions from the same oral appliance. For example, a light-emitting toothbrush can include two groups of LEDs, such that one group can radiate in a direction substantially parallel to the bristles, while the other group can radiate in the opposite or some other direction.

### Examples of Possible Treatments Using Embodiments According to Aspects of the Invention

Having described several embodiments according to aspects of the invention, it is clear that many different embodiments of photocosmetic devices are possible to treat various different conditions. The following is a discussion of examples of treatments that can be achieved using apparatus and methods according to aspects of the invention. However, the treatments discussed are exemplary and are not intended to be limiting. Apparatus and methods according the present invention are versatile and may be applied to known or yet-to-be-developed treatments.

Exemplary treatments include radiation-induced hair removal. Radiation-induced hair removal is a cosmetic treatment that could be performed by apparatus and methods according to aspects of the present invention. In the case of hair removal, the principal target for thermal damage or destruction is the hair bulb, including the matrix and papilla, and the stems cells around the hair bulge. For hair removal treatments, melanin located in the hair shaft and bulb is the targeted chromophore. While the bulb contains melanin and can thus be thermally treated, the basement membrane, which provides the hair growth communication pathway between the papilla within the bulb and the matrix within the hair shaft, contains the highest concentration of melanin and may be selectively targeted. Heating the hair shaft in the area of the bulge can cause thermal destruction of the stem cells surrounding the bulge.

Wavelengths between 0.6 and 1.2 μm are typically used for hair removal. By proper combination of power, speed, and focusing geometry, different hair related targets (e.g., bulb, matrix, basement membrane, stem cells) can be heated to the denaturation temperature while the surrounding dermis remains undamaged. Since the targeted hair follicle and the epidermis both contain melanin, a combination of epidermal contact cooling and long pulse width can be used to prevent epidermal damage. A more detailed explanation of hair removal is given in co-pending utility patent application Ser. No. 10/346,749, entitled “METHOD AND APPARATUS FOR HAIR GROWTH CONTROL,” by Rox Anderson, et al. filed Mar. 12, 2003, which is hereby incorporated herein by reference.

Hair removal is often required over large areas (e.g. back and legs), and the required power is therefore correspondingly large (on the order of 20-500 W) in order to achieve short treatment times. Current generation diode bars are capable of emitting 40-60 W at 800 nm, which makes them effective for use in some embodiments of a photocosmetic device according to the present invention.

Optionally, a topical lotion can be applied to the skin (e.g., via the handpiece) in a treatment area. In some embodiments, the transparent lotion is selected to have a refractive index in a range suitable to provide a waveguide effect to direct the light to a region of the skin to be irradiated. Preferably the index of refraction of the lotion is higher than the index of refraction of water (i.e., approximately 1.33 depending on chemical additives of the water). In some embodiments, the index of refraction of the lotion is higher than the index of refraction of the dermis (i.e., approximately 1.4). In some embodiments, the index of refraction of the lotion is higher than the index of refraction of the inner root sheath (i.e., approximately 1.55). In embodiments where the index of refraction is greater than the index of refraction of the inner root sheath, light incident on the surface of the skin can be delivered directly to hair matrix without significant attenuation.

The effective pulse length used to irradiate the skin is given by the beam size divided by the speed of scanning of the irradiation source. For example, a 2 mm beam size moved at a scanning speed of 50-100 mm/s provides an effective pulse length of 20-60 ms. For a power density of 250 W/cm the effective fluence is 5-10 J/cm², which approximately doubles the fluence of the light delivered by a device without the use of a high index lotion.

In some embodiments, the pH of the lotion can be adjusted to decrease the denaturation threshold of matrix cells. In such embodiments, lower power is required to injure the hair matrix and thus provide hair growth management. Optionally, the lotion can be doped by molecules or ions or atoms with significant absorption of light emitted by the source. Due to increased absorption of light in hair follicles when a suitable lotion is used, a lower power irradiation source may be used to provide sufficient irradiation to heat the hair matrix.

A second exemplary embodiment of a method of hair growth management according to the present invention includes first irradiating the skin, and then physically removing hair. By first irradiating the skin, attachment of the hair shaft to the follicle or the hair follicle to dermis is weakened. Consequently, mechanical or electromechanical depilation may be more easily achieved (e.g., by using a soft waxing or electromechanical epilator) and pain may be reduced.

Irradiation can weaken the attachment of the hair bulb to the skin or subcutaneous fat; therefore it is possible to pull out a significantly higher percentage of the hair follicle from the skin compared to the depilation alone. Because the diameter of the hair bulb is close to the diameter of the outer root sheath, pulling out hair with the hair bulb can permanently destroy the entire hair follicle including the associated stem cells. Accordingly, by first irradiating and then depilating, new hair growth can be decelerated or completely arrested.

Treatment of cellulite is another example of a cosmetic problem that may be treated by apparatus and methods according to aspects of the present invention. The formation of characteristic cellulite dimples begins with poor blood and lymph circulation, which in turn inhibits the removal of cellular waste products. For example, unremoved dead cells in the intracellular space may leak lipid over time. Connective tissue damage and subsequent nodule formation occurs due to the continuing accumulation of toxins and cellular waste products.

The following are two exemplary treatments for cellulite, both of which aim to stimulate both blood flow and fibroblast growth. In a first exemplary treatment, localized areas of thermal damage are created using a treatment source emitting in the near-infrared spectral range (e.g., at a wavelength in the range 650-1850 nm) in combination with an optical system designed to focus 2-10 mm beneath the skin surface. In one embodiment, light having a power density of 1-100 W/cm is delivered to the skin surface, and the apparatus is operated at a speed to create a temperature of 45 degrees Celsius at a distance 5 mm below the skin. The skin may be cooled to avoid or reduce damage to the epidermis to reduce wound formation. Further details of achieving a selected temperature a selected distance below the skin is given in U.S. patent application Ser. No. 09/634,691, filed Aug. 9, 2000, the substance of which was incorporated by reference herein above. The treatment may include compression of the tissue, massage of the tissue, or multiple passes over the tissue.

As noted above, acne is another very common skin disorder that can be treated using apparatus and methods according to aspects of the present invention. The following are additional exemplary methods of treating acne according to the present invention. In each of the exemplary methods, the actual treated area may be relatively small (assuming treatment of facial acne), thus a low-power CW source may be used.

A first possible treatment is to selectively damage the sebaceous gland to prevent sebum production. The sebaceous glands are located approximately 1 mm below the skin surface. By creating a focal spot at this depth and using a wavelength selectively absorbed by lipids (e.g., in proximity of 0.92, 1.2, and 1.7 μm), direct thermal destruction becomes possible. For example, to cause thermal denaturation, a temperature of 45-65 degrees Celsius may be generated at approximately 1 mm below the skin surface using any of the methods described in U.S. patent application Ser. No. 09/634,691, filed Aug. 9, 2000, the substance of which was incorporated by reference herein above.

An alternative treatment for acne involves heating a sebaceous gland to a point below the thermal denaturation temperature (e.g., to a temperature 45-65 degrees Celsius) to achieve a cessation of sebum production and apoptosis (programmed cell death). Such selective treatment may take advantage of the low thermal threshold of cells responsible for sebum production relative to surrounding cells.

Another alternative treatment of acne is thermal destruction of the blood supply to the sebaceous glands (e.g., by heating the blood to a temperature 60-95 degrees Celsius).

For the above treatments of acne, the sebaceous gland may be sensitized to near-infrared radiation by using compounds such as indocyanine green (ICG, absorption near 800 nm) or methylene blue (absorption near 630 nm). Alternatively, non-thermal photodynamic therapy agents such as photofrin may be used to sensitize sebaceous glands. In some embodiments, biochemical carriers such as monoclonal antibodies (MABs) may be used to selectively deliver these sensitization compounds directly to the sebaceous glands.

Although the above procedures were described as treatments for acne, because the treatments involve damage/destruction of the sebaceous glands (and therefore reduction of sebum output), the treatments may also be used to treat excessively oily skin.

Yet another technique for treating acne involves using light to expand the opening of an infected hair follicle to allow unimpeded sebum outflow. In one embodiment of the technique, a lotion that preferentially accumulates in the follicle opening (e.g., lipid consistent lotion with organic non organic dye or absorption particles) is applied to the skin surface. A treatment source wavelength is matched to an absorption band of the lotion. For example, in the case of ICG doped lotion the source wavelength is 790-810 nm By using an optical system to generate a temperature of 45-100 degrees Celsius at the infundibulum/infrainfundibulum, for example, by generating a fluence of at skin surface (e.g., 1-100 W/cm), the follicle opening can be expanded and sebum is allowed to flow out of the hair follicle and remodeling of infrainfundibulum in order to prevent comedo (i.e., blackhead) formation.

Non-ablative wrinkle treatment, which is now used as an alternative to traditional ablative CO₂ laser skin resurfacing, is another cosmetic treatment that could be performed by apparatus and methods according to aspects of the present invention. Non-ablative wrinkle treatment is achieved by simultaneously cooling the epidermis and delivering light to the upper layer of the dermis to thermally stimulate fibroblasts to generate new collagen deposition.

An embodiment of a photocosmetic device could include a sensor that will detect fluorescence in newer collagen in the skin by shining light on the skin in the blue range, in particular approximately 380-390 nm.

In wrinkle treatment, because the primary chromophore is water, wavelengths ranging from 0.8-2 μm are appropriate wavelengths for use in the treatment. Since only wrinkles on the face are typically of cosmetic concern, the treated area is typically relatively small and the required coverage rate (cm²/sec) is correspondingly low, and a relatively low-power treatment source may be used. An optical system providing sub-surface focusing in combination with epidermal cooling may be used to achieve the desired result. Precise control of the upper-dermis temperature is important; if the temperature is too high, the induced thermal damage of the epidermis will be excessive, and if the temperature is too low, the amount of new collagen deposition will be minimal. A speed sensor (in the case of a manually scanned handpiece) or a mechanical drive may be used to precisely control the upper-dermis temperature. Alternatively, a non-contact mid-infrared thermal sensor could be used to monitor dermal temperature.

Pigmented lesions such as age spots can be removed by selectively targeting the cells containing melanin in these structures. These lesions are located using an optical system focusing at a depth of 100-200 μm below the skin surface and can be targeted with wavelengths in the 0.4-1.1 μm range. Since the individual melanin-bearing cells are small with a short thermal relaxation time, a shallow sub-surface focus is helpful to reach the denaturation temperature.

Elimination of underarm odor is another problem that could be treated by an apparatus and methods according to aspects of the present invention. In such a treatment, a source having a wavelength selectively absorbed by the eccrine/apocrine glands is used to thermally damage the eccrine/apocrine glands. Optionally, a sensitization compound may be used to enhance damage.

Absorption of light by a chromophore within a tissue responsible for an unwanted cosmetic condition or by a chromophore in proximity to the tissue could also be performed using embodiments according to aspects of the present invention. Treatment may be achieved by limited heating of the target tissue below temperature of irreversible damage or may be achieved by heating to cause irreversible damage (e.g., denaturation). Treatment may be achieved by direct stimulation of biological response to heat, or by induction of a cascade of phenomena such that a biological response is indirectly achieved by heat. A treatment may result from a combination of any of the above mechanisms. Optionally, cooling, DC or AC(RF) electrical current, physical vibration or other physical stimulus may be applied to a treatment area or adjacent area to increase the efficacy of a treatment. A treatment may require a single session, or multiple sessions may be used to achieve a desired effect.

Having thus described the inventive concepts and a number of exemplary embodiments, it will be apparent to those skilled in the art that the invention may be implemented in various ways, and that modifications and improvements will readily occur to such persons. Thus, the examples given are not intended to be limiting. Also, it is to be understood that the use of the terms “including,” “comprising,” or “having” is meant to encompass the items listed thereafter and equivalents thereof as well as additional items before, after, or in-between the items listed.

What is claimed is:

1. A handheld device for the treatment of acne using radiant energy, comprising:
a housing having an aperture;
a radiation source mounted in said housing and oriented to transmit radiation through said aperture; and
a heat dissipation element mounted in said housing and in thermal communication with said radiation source;
wherein said radiation source is configured to irradiate said tissue with radiation between approximately 10 mW/cm² and approximately 100 W/cm².

2. The handheld device of claim 1, wherein said radiation source is configured to irradiate said tissue with radiation between approximately 100 mW/cm and approximately 100 W/cm².

3. The handheld device of claim 1, wherein said radiation source is configured to irradiate said tissue with radiation between approximately 1 W/cm² and approximately 100 W/cm².

4. The handheld device of claim 1, wherein said radiation source is configured to irradiate said tissue with radiation between approximately 10 W/cm and approximately 100 W/cm².

5. The handheld device of claim 1, wherein said aperture has an area of at least approximately 4 cm.

6. The handheld device of claim 1, wherein said aperture has an area of at least approximately 9 cm².

7. The handheld device of claim 1, wherein said aperture has an area of at least approximately 14.44 cm².

8. The handheld device of claim 1, wherein said aperture has an area of at least approximately 16 cm².

9. The handheld device of claim 1, wherein said radiation source is configured to provide at least approximately 2.5 W of optical power.

10. The handheld device of claim 1, wherein said radiation source is configured to provide at least approximately 5 W of optical power.

11. The handheld device of claim 1, wherein said radiation source is configured to provide at least approximately 10 W of optical power.

12. The handheld device of claim 1, wherein said handheld device is a device for self-use by a consumer.

13. The handheld device of claim 1, wherein said housing has a head portion containing said aperture and a handle portion configured to be held by a user to allow the aperture to be moved over the tissue as optical radiation is generated by the radiation source.

14. The handheld device of claim 1, wherein said aperture includes a sapphire window.

15. The handheld device of claim 1, wherein said aperture includes a plastic window.

16. The handheld device of claim 1, wherein said radiation source is a solid state optical radiation source.

17. The handheld device of claim 1, wherein said radiation source is an LED radiation source.

18. The handheld device of claim 1, wherein said radiation source is a laser radiation source.

19. The handheld device of claim 1, wherein said radiation source is an array of semiconductor elements.

20. The handheld device of claim 1, wherein said radiation source is an optical radiation source.

21. The handheld device of claim 1, wherein said radiation source is a first radiation source and said device further includes a second radiation source, wherein said first radiation source is capable of generating radiation having a wavelength within a first range of wavelengths and said second radiation source is capable of generating radiation having a wavelength within a second range of wavelengths.

22. The handheld device of claim 21, wherein said first and second ranges of wavelengths do not overlap.

23. The handheld device of claim 21, further comprising a power source;
wherein said first radiation source is electrically connected to said power source along a first electrical connection path, and said second radiation source is electrically connected to said power source along a second electrical connection path such that the first radiation source is capable of producing radiation independently from said second radiation source.

24. The handheld device of claim 1, wherein said radiation source is an array of semiconductor elements.

25. The handheld device of claim 1, wherein said radiation source is operable at multiple wavelengths.

26. The handheld device of claim 1, further comprising a power source.

27. The handheld device of claim 26, wherein said power source is configured to supply power in a continuous wave mode.

28. The handheld device of claim 26, wherein said power source is configured to supply power in a quasi-continuous wave mode.

29. The handheld device of claim 26, wherein said power source is configured to supply power in a pulsed wave mode.

30. The handheld device of claim 1, further comprising a first sensor electrically connected to a controller, said first sensor configured to provide a first electrical signal when a first section of said aperture is in contact with said tissue, said controller causing said radiation source to be illuminated when said sensor provides said first electrical signal.

31. The handheld device of claim 30, wherein the radiation source comprises a first radiation source and the device further comprises a second radiation source and a second sensor electrically connected to said controller, said second sensor configured to provide a second electrical signal when a second portion of said aperture is in contact with said tissue, said controller causing said second radiation source to be illuminated when said sensor provides said second electrical signal.

32. The handheld device of claim 1, wherein said radiation source is an array of solid state optical radiation sources.

33. The handheld device of claim 1, wherein said aperture is thermally conductive, said radiation source being directly adjacent to said aperture such that said aperture provides a third thermal conduction path allowing heat from said radiation source to be transferred to an area of said tissue being treated via said aperture.

34. The handheld device of claim 1, wherein said housing further includes an alarm electrically connected to said controller; said controller configured to provide an output signal to said alarm to provide information to said user.

35. The handheld device of claim 34, wherein said alarm is an audible sound generator.

36. The handheld device of claim 34 wherein said alarm is a light-emitting device.

37. The handheld device of claim 34, wherein said alarm is configured to alert the user that a treatment time has expired.

38. A handheld device for the treatment of acne using radiant energy, comprising:
a housing having an aperture;
a radiation source oriented to transmit radiation through said aperture;
a controller electrically connected to said radiation source;
a sensor electrically connected to said controller, wherein said controller is configured to provide an output signal in response to an input signal from said sensor; and
wherein said radiation source is configured to irradiate said tissue with radiation between approximately 1 W/cm and approximately 100 W/cm².
